(19) United States
(12) Patent Application Publication
(10) Pub. No.: US 2024/0342311 A1
(43) Pub. Date: Oct. 17, 2024

(54) ENGINEERING AAV VECTORS WITH IMPROVED CNS TARGETING

(71) Applicant: University of Florida Research Foundation, Incorporated, Gainesville, FL (US)

(72) Inventors: Robert McKenna, Gainesville, FL (US); Mavis Agbandje-Mckenna, Gainesville, FL (US); Judit Penzes, Gainesville, FL (US); Mario Mietzsch, Gainesville, FL (US)

(73) Assignee: University of Florida Research Foundation, Incorporated, Gainesville, FL (US)

(21) Appl. No.: 18/293,040

(22) PCT Filed: Jul. 29, 2022
(86) PCT No.: PCT/US2022/074316

§ 371 Date: Jan. 29, 2024

Related U.S. Application Data
(60) Provisional application No. 63/227,959, filed on Jul. 30, 2021.

(51) Int. Cl. A61K 48/00; C12N 15/86

(52) U.S. Cl. CPC A61K 48/0058; C12N 15/86; C12N 2750/14122; C12N 2750/14143; C12N 2750/14145

(57) ABSTRACT

Provided herein are modified adeno-associated virus (AAV) capsid proteins, particles, nucleic acid vectors, and compositions thereof, as well as methods of their use, such as in the treatment of neurological diseases and disorders. Specifically, the disclosure provides a modified AAV capsid protein comprising a non-native peptide within loop IV or loop VIII of the capsid protein. Further provided are non-native peptide sequences for insertion.

### RELATED APPLICATIONS

This application claims priority under 35 U.S.C. § 119(e) to U.S. Provisional Application Ser. No. 63/227,959, entitled “ENGINEERING AAV VECTORS WITH IMPROVED CNS TARGETING”, filed Jul. 30, 2021, the entire contents of which are herein incorporated by reference.

### REFERENCE TO AN ELECTRONIC SEQUENCE LISTING

The contents of the electronic sequence listing (U119670087WO00-SEQ-COB.xml; Size: 36,476 bytes; and Date of Creation: Jul. 27, 2022) are herein incorporated by reference in their entirety.

### BACKGROUND OF THE INVENTION

Gene therapy has the potential to treat subject suffering from or are at risk of suffering from various diseases and conditions. Improved adeno-associated virus (AAV) vectors for carrying payloads would be beneficial to the development of gene therapies, e.g., for certain diseases that affect neurological (e.g., central nervous system (CNS)) tissue and/or function.

Neurological diseases can result from numerous conditions including, for example, congenital or acquired somatic mutations, injury, and exposure to hazardous compounds. In some cases, neurological diseases result in life-threatening complications or lead to serious symptoms and/or death. Although numerous factors have been implicated in regulating neurological diseases, effective treatments remain limited.

### SUMMARY OF THE INVENTION

The present disclosure is based at least in part on the realization that certain amino acid substitutions in one or more capsid proteins of a recombinant AAV (rAAV) particle confer improved properties (e.g., targeting to particular tissues and transduction of particular cell types) to the rAAV particle relative to a corresponding wild-type (e.g., not comprising the modified capsid protein) particle. In addition, substitution of particular amino acids by certain peptides or insertion of certain peptides at particular positions within one or more capsid proteins of an rAAV particle confers improved properties to the rAAV particle relative to a corresponding wild-type particle.

According to some aspects, modified adeno-associated virus (AAV) capsid proteins are provided. In some embodiments, a modified AAV capsid protein comprises a non-native peptide within loop IV or loop VIII of the capsid protein, wherein the non-native peptide comprises an amino acid sequence selected from GRILARGEINFK (SEQ ID NO: 15), ASKKPKRNIKA (SEQ ID NO: 16), AKKMWKKTW (SEQ ID NO: 17), GEISVGESKFFL (SEQ ID NO: 18), KHIFSDDSSELTIRNVDKNDE (SEQ ID NO: 19), and SIHLKVFAK (SEQ ID NO: 20). In some embodiments, a modified AAV capsid protein comprises an arginine amino acid at a position corresponding to F535 of the wild-type AAV1 capsid protein as set forth in SEQ ID NO: 1.

In some embodiments, the non-native peptide is positioned between amino acids corresponding to Q450 and K459 of the wild-type AAV1 capsid protein as set forth in SEQ ID NO: 1. In some embodiments, the non-native peptide replaces the amino acids corresponding to positions 451 to 458 of the wild-type AAV1 capsid protein as set forth in SEQ ID NO: 1. In some embodiments, the non-native peptide is positioned between amino acids corresponding to S587 and S588 of the wild-type AAV1 capsid protein as set forth in SEQ ID NO: 1.

In some embodiments, the modification results in increased binding of the AAV capsid protein to glycans relative to a corresponding unmodified AAV capsid protein. In some embodiments, the modification results in increased binding of the AAV capsid protein to polysialic acid relative to a corresponding unmodified AAV capsid protein. In some embodiments, the polysialic acid is attached to a neuronal cell adhesion molecule.

In some embodiments, the modification results in increased binding of the AAV capsid protein to a cell of the central nervous system relative to a corresponding unmodified AAV capsid protein. In some embodiments, the cell of the central nervous system is a neuronal cell. In some embodiments, the neuronal cell is a brain neuronal cell.

In some embodiments, the AAV capsid protein is an AAV1, AAV9, AAVrh10 serotype capsid protein, or a combination thereof.

According to some aspects, AAV particles comprising a modified AAV capsid protein disclosed herein are provided.

In some embodiments, the AAV particle is less immunogenic relative to a corresponding AAV particle not comprising the modified capsid protein.

In some embodiments, the AAV particle is less susceptible to binding by a neutralizing antibody than a corresponding wild-type AAV particle not comprising the modified capsid protein. In some embodiments, the neutralizing antibody is an ADKla, ADK1b, ADK9, HL2370, HL2374, ADK8, HL2381, or HL2383 antibody.

In some embodiments, the transduction efficiency of the AAV particle in a cell of the central nervous system is increased relative to a corresponding AAV particle not comprising the modified capsid protein.

In some embodiments, the AAV particle further comprises a nucleic acid segment encoding a therapeutic or diagnostic agent operably linked to a promoter. In some embodiments, the promoter is a GAD65 promoter, a SYN promoter, a GFAP promoter, an INA promoter, an NES promoter, an MOBP promoter, an MBP promoter, a TH promoter, or a FOXA2/HNF30 promoter. In some embodiments, the therapeutic or diagnostic agent is IDUA, IDS, SGSH, NAGLU, TPP1/CLN2, CLN3, CLN6, ARSA, ASPA, AADC, NTN, GDNF, NGF, APOE2, or SMN.

In some embodiments, the AAV particle comprises the modified AAV capsid protein and an unmodified wild-type capsid protein. In some embodiments, the ratio of unmodified wild-type capsid proteins to modified capsid proteins is about 95:5, 90:10, 85:15, 80:20, 75:25, 70:30, 66:33, 50:50; 33:66, 25:75, 20:80, 15:85, 10:90, or 5:95.

In some embodiments, the particle is an AAV1, AAV9, or AAVrh10 serotype particle, or a combination thereof.

According to some aspects, methods of delivering a therapeutic or diagnostic agent to a cell are provided. In some embodiments, a method of delivering a therapeutic or diagnostic agent to a cell comprises contacting a cell with an AAV particle disclosed herein in an amount sufficient to promote internalization of the AAV particle in the cell, optionally wherein the cell is a central nervous system cell.

According to some aspects, methods of treating a subject are provided. In some embodiments, a method of treating a subject comprises administering an AAV particle disclosed herein to a subject in need thereof in an amount sufficient to alleviate one or more symptoms in the subject.

In some embodiments, the subject is suspected of having or has been diagnosed as having a neurological disease, disorder, or condition. In some embodiments, the subject is a mammal. In some embodiments, the subject is a human.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1D show grafting of residues from Minute virus of mice strain i (MVMi) onto AAV1. FIG. 1A shows the binding site of GT3 glycan to the capsid of MVMi as determined by X-ray crystallography. The elongated filled oval shows the position of the 2-fold symmetry axis of the MVMi capsid. The different outlined regions indicate the different monomers of the capsid. FIG. 1B shows the structure of GT3, which is a glycan molecule with a terminal polysialic acid chain. The filled diamonds represent sialic acids, empty circle represents galactose, filled circle represents glucose. FIG. 1C shows an atomic model of MVMi and GT3 at the polysialic acid binding site. The R368 residue of MVMi structurally aligns with F535 of AAV1. FIG. 1D shows the transduction efficiency of AAV1 wild-type and F535R capsid variants in the neuronal cell lines Neuro2A (left) and U87 (right). The results were normalized to the transduction of wild-type AAV1. The experiment was performed in triplicate (n=3) and is shown as mean+standard deviation.

FIGS. 2A-2B shows the position of the F535R amino acid substitution in the AAV1 capsid protein. FIG. 2A shows the VP protein structure as a ribbon diagram viewed from the side. The positions of the surface variable regions (VRs) and F535 (of the wild-type protein) and R535 (of the engineered protein) are labeled. FIG. 2B shows a surface representation of the AAV1 capsid with the 2-, 3-, and 5-fold symmetry axes shown. The position of R535 at the side of the 3-fold protrusions is indicated with arrows and is shaded on the capsid itself.

FIGS. 3A-3C show engineering of AAV capsid proteins for targeting of neuronal cell adhesion molecule (NCAM). FIG. 3A shows superposed VP structures of AAV1 wild-type capsid protein (AAV1-WT), VR-IV-P2 modified capsid protein (AAV1-VR-IV-P2) or VR-VIII-P2 modified capsid protein (AAV1-VR-VIII-P2) as ribbon diagrams viewed from the side. The positions of the surface variable regions (VRs) are indicated. The P2 peptide in VR-IV-P2 replaces amino acids 451-458 of the wild-type AAV1 capsid protein sequence, resulting in a 4 amino acid longer loop relative to the AAV1-WT capsid protein. In VR-VIII-P2, the P2 peptide is inserted between amino acids 587 and 588 according to the wild-type AAV1 sequence numbering, resulting in a 12 amino acid insertion. FIG. 3B shows the amino acid sequence alignment of the AAV1 capsid variants at the P2 insertion loci. FIG. 3C shows the structure of NCAM as expressed in the brain. Its Ig-like domains are glycosylated with poly-sialylated glycans.

FIGS. 4A-4B show transduction efficiency of wild-type AAV1, AAV1 F535R capsid variant, AAV1-VR-IV-P2 capsid variant, and AAV1-VR-VIII-P2 capsid variant in the neuronal cell lines Neuro2A (FIG. 4A) and U87 (FIG. 4B). The results were normalized to the transduction of wild-type AAV1. The experiment was performed in triplicate (n=3) and results are shown as mean+standard deviation.

FIGS. 5A-5B show transduction efficiency of AAV9 capsid variants having varying ratios of wild-type and VR-IV-P2 and VR-VIII-P2 peptide modified capsid proteins in neuronal cell lines U87 (FIG. 5A) and Neuro2A (FIG. 5B). The experiment was performed in triplicate (n=3) and results are shown as mean+standard deviation relative luminescence units relative to wild-type AAV9. NT indicates the capsid variant with the indicated ratio of wild-type to P2 modified capsid proteins was not tested due to low yield of construct.

FIGS. 6A-6B show transduction efficiency of AAVrh.10 capsid variants having varying ratios of wild-type and VR-IV-P2 and VR-VIII-P2 peptide modified capsid proteins in neuronal cell lines U87 (FIG. 6A) and Neuro2A (FIG. 6B). The experiment was performed in triplicate (n=3) and results are shown as mean+standard deviation relative luminescence units relative to wild-type AAVrh.10. NT indicates the capsid variant with the indicated ratio of wild-type to P2 modified capsid proteins was not tested due to low yield of construct.

FIG. 7 shows results of an analysis of the antigenicity of AAV P2 peptide capsid variants, evaluated based on binding of capsids immobilized on a nitrocellulose membrane by monoclonal antibodies (MAbs) ADKla, ADK1b (for AAV1 variants; left panel), ADK8, HL2368, HL2370, HL2372, HL2374 (for AAV9 variants; middle panel), and ADK8, ADK8/9, HL2381, HL2383 (for AAVrh.10 variants; right panel), which are specific for conformational epitopes of AAV capsid proteins. MAb B 1 was used as a positive control for denatured capsids.

FIGS. 8A-8C show density maps of AAV9 capsids reconstructed from cryo-electron microscopy (cryo-EM) data. The approximate locations of VR-IV and VR-VIII are labeled in each density map with a dotted circle and a dotted rectangle, respectively. FIG. 8A shows a cryo-EM reconstructed density map of an AAV9 variant capsid comprising capsid proteins with P2 peptide inserted in VR-IV (inserted between amino acids 1451 and Q459, and replacing amino acids 452-458). FIG. 8B shows a cryo-EM reconstructed density map of a wild-type AAV9 capsid. FIG. 8C shows a cryo-EM reconstructed density map of an AAV9 variant capsid comprising capsid proteins with P2 peptide inserted in VR-VIII (inserted between amino acids Q588 and A589).

FIGS. 9A-9B show atomic structures of AAV9-P2 variants. Each structure shows an electron density map fitted with models of VR-IV (FIG. 9A) or VR-VIII (FIG. 9B) contoured to 1σ. Nitrogen atoms are labeled with *; oxygen atoms are labeled with #; and carbon atoms are shown without a label.

FIGS. 10A-10C show density maps of AAVrh.10 capsids reconstructed from cryo-electron microscopy (cryo-EM) data. The approximate locations of VR-IV and VR-VIII are labeled in each density map with a dotted circle and a dotted rectangle, respectively. FIG. 10A shows a cryo-EM reconstructed density map of an AAVrh.10 variant capsid comprising capsid proteins with P2 peptide inserted in VR-IV (inserted between amino acids Q452 and Q461, and replacing amino acids 453-460). FIG. 10B shows a cryo-EM reconstructed density map of a wild-type AAVrh.10 capsid. FIG. 10C shows a cryo-EM reconstructed density map of an AAVrh.10 variant capsid comprising capsid proteins with P2 peptide inserted in VR-VIII (inserted between amino acids Q589 and N590).

FIGS. 11A-11B show atomic structures of AAVrh.10-P2 variants. Each structure shows an electron density map fitted with models of VR-IV (FIG. 11A) or VR-VIII (FIG. 11B) contoured to 1σ. Nitrogen atoms are labeled with *; oxygen atoms are labeled with #; and carbon atoms are shown without a label.

### DETAILED DESCRIPTION

The following detailed description is made by way of illustration of certain aspects of the disclosure. It is to be understood that other aspects are contemplated and may be made without departing from the scope or spirit of the present disclosure. The following detailed description, therefore, is not to be taken in a limiting sense. Scientific and technical terms used herein have meanings commonly used in the art unless otherwise specified. The definitions provided herein are to facilitate understanding of certain terms used frequently herein and are not meant to limit the scope of the present disclosure.

The present disclosure is based at least in part on the development of adeno-associated virus (AAV) capsid proteins, particles, genomes, nucleic acid vectors, and plasmids useful in the delivery of various cargoes to particular cells, facilitating transgene expression therein. The AAV capsid proteins, particles, genomes, nucleic acid vectors, and plasmids disclosed herein may be used in a variety of applications including but not limited to compositions and methods (e.g., therapeutic methods). Therapeutic methods disclosed herein include those useful in the treatment of diseases (e.g., neurological disorders, such as those affecting the CNS), in subjects in need thereof.

Provided herein are compositions, including AAV capsid proteins comprising modifications (e.g., amino acid substitutions or insertions of peptides), AAV particles, nucleic acids comprised within AAV particles, and methods of using the compositions for transducing a cell of interest (e.g., for treating a disease or condition in a subject).

### AAV Structure

The AAV genome is built of single-stranded deoxyribonucleic acid (ssDNA), which is either positive- or negative-sensed. At each end of the DNA strand is an inverted terminal repeat (ITR). Between the ITRs are two open reading frames (ORFs): rep and cap. The rep ORF is composed of four overlapping genes encoding Rep proteins required for the AAV life cycle. The cap ORF contains overlapping nucleotide sequences of capsid proteins: VP1, VP2 and VP3, which interact together to form a capsid of an icosahedral symmetry.

The capsid proteins, which are controlled by the same promoter, designated p40, are translated from the same mRNA. The molecular weights of VP1, VP2 and VP3 are 87, 72 and 62 kiloDaltons (kDa), respectively. The AAV capsid is composed of 60 capsid protein subunits, VP1, VP2, and VP3, that are arranged in an icosahedral symmetry in a ratio of 1:1:10.

An AAV capsid protein disclosed herein can be of any serotype, or can be a chimeric capsid protein (i.e., comprising segments from capsid proteins of two or more serotypes). In some embodiments, a capsid protein disclosed herein is an AAV1, AAV2, AAV3, AAV4, AAV5, AAV6, AAV7, AAV8, AAV9, AAV10, AAV11, AAV12, AAV13, or AAVrh10 capsid protein. In some embodiments, an AAV capsid protein as provided herein is of serotype 1, 9, or rh10. In some embodiments, the AAV capsid protein is an AAV1 capsid protein. In some embodiments, the AAV capsid protein is an AAV9 capsid protein. In some embodiments, the AAV capsid protein is an AAVrh10 capsid protein. Amino acid sequences of capsid proteins of other AAV serotypes are known and can be aligned with SEQ ID NO: 1 (AAV1 capsid protein) using techniques known in the art.

In some embodiments, an AAV capsid protein as disclosed herein is a VP1 protein, a VP2 protein, or a VP3 protein. The VP1, VP2, and VP3 capsid proteins are each encoded from the same segment of the AAV genome, and differ in their N termini based on alternative mRNA splicing.

Non-limiting examples of wild-type AAV capsid protein sequences are provided below.

### Example of Wild-Type AAV1 Capsid Protein Sequence

| (SEQ ID NO: 1) | |
|---|---|
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTINDGV TTIANNLIST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEEVP FHSSYAHSQS LDRLMNPLID QYLYYLNRTQ |
| 451 | NQSGSAQNKD LLFSRGSPAG MSVQPKNWLP GPCYRQQRVS KTKTDNNNSN |
| 501 | FTWTGASKYN LNGRESIINP GTAMASHKDD EDKFFPMSGV MIFGKESAGA |
| 551 | SNTALDNVMI IDEEEIKATN PVATERFGTV AVNFQSSSTD PATGDVHAMG |
| 601 | ALPGMVWQDR DVYLQGPIWA KIPHTDGHFH PSPLMGGFGL KNPPPQILIK |
| 651 | NTPVPANPPA EFSATKFASF ITQYSTGQVS VEIEWELQKE NSKRWNPEVQ |
| 701 | YTSNYAKSAN VDFTVDNNGL YTEPRPIGTR YLTRPL |

### Example of Wild-Type AAV2 Capsid Protein Sequence

| (SEQ ID NO: 2) | |
|---|---|
| 1 | MAADGYLPDW LEDTLSEGIR QWWKLKPGPP PPKPAERHKD DSRGLVLPGY |
| 51 | KYLGPFNGLD KGEPVNEADA AALEHDKAYD RQLDSGDNPY LKYNHADAEF |
| 101 | QERLKEDTSF GGNLGRAVFQ AKKRVLEPLG LVEEPVKTAP GKKRPVEHSP |
| 151 | VEPDSSSGTG KAGQQPARKR LNFGQTGDAD SVPDPQPLGQ PPAAPSGLGT |
| 201 | NTMATGSGAP MADNNEGADG VGNSSGNWHC DSTWMGDRVI TTSTRTWALP |
| 251 | TYNNHLYKQI SSQSGASNDN HYFGYSTPWG YFDFNRFHCH FSPRDWQRLI |
| 301 | NNNWGFRPKR LNFKLFNIQV KEVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFT FSYTFEDVPF HSSYAHSQSL DRLMNPLIDQ YLYYLSRINT |
| 451 | PSGTTTQSRL QFSQAGASDI RDQSRNWLPG PCYRQQRVSK TSADNNNSEY |
| 501 | SWTGATKYHL NGRDSLVNPG PAMASHKDDE EKFFPQSGVL IFGKQGSEKT |
| 551 | NVDIEKVMIT DEEEIRTTNP VATEQYGSVS TNLQRGNRQA ATADVNTQGV |
| 601 | LPGMVWQDRD VYLQGPIWAK IPHTDGHFHP SPLMGGFGLK HPPPQILIKN |
| 651 | TPVPANPSTT FSAAKFASFI TQYSTGQVSV EIEWELQKEN SKRWNPEIQY |
| 701 | TSNYNKSVNV DFTVDINGVY SEPRPIGTRY LTRNL |

### Example of Wild-Type AAV3 Capsid Protein Sequence

| (SEQ ID NO: 3) | |
|---|---|
| 1 | MAADGYLPDW LEDNLSEGIR EWWALKPGVP QPKANQQHQD NRRGLVLPGY |
| 51 | KYLGPGNGLD KGEPVNEADA AALEHDKAYD QQLKAGDNPY LKYNHADAEF |
| 101 | QERLQEDTSF GGNLGRAVFQ AKKRILEPLG LVEEAAKTAP GKKGAVDQSP |
| 151 | QEPDSSSGVG KSGKQPARKR LNFGQTGDSE SVPDPQPLGE PPAAPTSLGS |
| 201 | NTMASGGGAP MADNNEGADG VGNSSGNWHC DSQWLGDRVI TTSTRTWALP |
| 251 | TYNNHLYKQI SSQSGASNDN HYFGYSTPWG YFDFNRFHCH FSPRDWQRLI |
| 301 | NNNWGFRPKK LSFKLFNIQV RGVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFQ FSYTFEDVPF HSSYAHSQSL DRLMNPLIDQ YLYYLNRTQG |
| 451 | TTSGTTNQSR LLFSQAGPQS MSLQARNWLP GPCYRQQRLS KTANDNNNSN |
| 501 | FPWTAASKYH LNGRDSLVNP GPAMASHKDD EEKFFPMHGN LIFGKEGTTA |
| 551 | SNAELDNVMI TDEEEIRTIN PVATEQYGTV ANNLQSSNTA PTTGTVNHQG |
| 601 | ALPGMVWQDR DVYLQGPIWA KIPHTDGHFH PSPLMGGFGL KHPPPQIMIK |
| 651 | NTPVPANPPT TFSPAKFASF ITQYSTGQVS VEIEWELQKE NSKRWNPEIQ |
| 701 | YTSNYNKSVN VDFTVDINGV YSEPRPIGTR YLTRNL |

### Example of Wild-Type AAV4 Capsid Protein Sequence

| (SEQ ID NO: 4) | |
|---|---|
| 1 | MTDGYLPDWL EDNLSEGVRE WWALQPGAPK PKANQQHQDN ARGLVLPGYK |
| 51 | YLGPGNGLDK GEPVNAADAA ALEHDKAYDQ QLKAGDNPYL KYNHADAEFQ |
| 101 | QRLQGDTSFG GNLGRAVFQA KKRVLEPLGL VEQAGETAPG KKRPLIESPQ |
| 151 | QPDSSTGIGK KGKQPAKKKL VFEDETGAGD GPPEGSTSGA MSDDSEMRAA |
| 201 | AGGAAVEGGQ GADGVGNASG DWHCDSTWSE GHVITTSTRT WVLPTYNNHL |
| 251 | YKRLGESLQS NTYNGFSTPW GYFDFNRFHC HFSPRDWQRL INNNWGMRPK |
| 301 | AMRVKIFNIQ VKEVTTSNGE TTVANNLIST VQIFADSSYE LPYVMDAGQE |
| 351 | GSLPPFPNDV FMVPQYGYCG LVTGNTSQQQ TDRNAFYCLE YFPSQMLRTG |
| 401 | NNFEITYSFE KVPFHSMYAH SQSLDRLMNP LIDQYLWGLQ STTTGTTLNA |
| 451 | GTATTNFTKL RPTNFSNFKK NWLPGPSIKQ QGFSKTANQN YKIPATGSDS |
| 501 | LIKYETHSTL DGRWSALTPG PPMATAGPAD SKFSNSQLIF AGPKQNGNTA |
| 551 | TVPGTLIFTS EEELAATNAT DTDMWGNLPG GDQSNSNLPT VDRLTALGAV |
| 601 | PGMVWQNRDI YYQGPIWAKI PHTDGHFHPS PLIGGFGLKH PPPQIFIKNT |
| 651 | PVPANPATTF SSTPVNSFIT QYSTGQVSVQ IDWEIQKERS KRWNPEVQFT |
| 701 | SNYGQQNSLL WAPDAAGKYT EPRAIGTRYL THHL |

### Example of Wild-Type AAV5 Capsid Protein Sequence

| (SEQ ID NO: 5) | |
|---|---|
| 1 | MSFVDHPPDW LEEVGEGLRE FLGLEAGPPK PKPNQQHQDQ ARGLVLPGYN |
| 51 | YLGPGNGLDR GEPVNRADEV AREHDISYNE QLEAGDNPYL KYNHADAEFQ |
| 101 | EKLADDTSFG GNLGKAVFQA KKRVLEPFGL VEEGAKTAPT GKRIDDHFPK |
| 151 | RKKARTEEDS KPSTSSDAEA GPSGSQQLQI PAQPASSLGA DTMSAGGGGP |
| 201 | LGDNNQGADG VGNASGDWHC DSTWMGDRVV TKSTRTWVLP SYNNHQYREI |
| 251 | KSGSVDGSNA NAYFGYSTPW GYFDFNRFHS HWSPRDWQRL INNYWGFRPR |
| 301 | SLRVKIFNIQ VKEVTVQDST TTIANNLIST VQVFTDDDYQ LPYVVGNGTE |
| 351 | GCLPAFPPQV FTLPQYGYAT LNRDNTENPT ERSSFFCLEY FPSKMLRTGN |
| 401 | NFEFTYNFEE VPFHSSFAPS QNLFKLANPL VDQYLYRFVS TNNTGGVQFN |
| 451 | KNLAGRYANT YKNWFPGPMG RTQGWNLGSG VNRASVSAFA TTNRMELEGA |
| 501 | SYQVPPQPNG MTNNLQGSNT YALENTMIFN SQPANPGTTA TYLEGNMLIT |
| 551 | SESETQPVNR VAYNVGGQMA INNQSSTTAP ATGTYNLQEI VPGSVWMERD |
| 601 | VYLQGPIWAK IPETGAHFHP SPAMGGFGLK HPPPMMLIKN TPVPGNITSF |
| 651 | SDVPVSSFIT QYSTGQVIVE MEWELKKENS KRWNPEIQYT NNYNDPQFVD |
| 701 | FAPDSTGEYR TTRPIGTRYL TRPL |

### Example of Wild-Type AAV6 Capsid Protein Sequence

| (SEQ ID NO: 6) | |
|---|---|
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPFG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTINDGV TTIANNLIST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEDVP FHSSYAHSQS LDRLMNPLID QYLYYLNRTQ |
| 451 | NQSGSAQNKD LLFSRGSPAG MSVQPKNWLP GPCYRQQRVS KTKTDNNNSN |
| 501 | FTWTGASKYN LNGRESIINP GTAMASHKDD KDKFFPMSGV MIFGKESAGA |
| 551 | SNTALDNVMI TDEEEIKATN PVATERFGTV AVNLQSSSTD PATGDVHVMG |
| 601 | ALPGMVWQDR DVYLQGPIWA KIPHTDGHFH PSPLMGGFGL KHPPPQILIK |
| 651 | NTPVPANPPA EFSATKFASF ITQYSTGQVS VEIEWELQKE NSKRWNPEVQ |
| 701 | YTSNYAKSAN VDFTVDNNGL YTEPRPIGTR YLTRPL |

### Example of Wild-Type AAV7 Capsid Protein Sequence

| (SEQ ID NO: 7) | |
|---|---|
| 1 | MAADGYLPDW LEDNLSEGIR EWWDLKPGAP KPKANQQKQD NGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP AKKRPVEPSP |
| 151 | QRSPDSSTGI GKKGQQPARK RLNFGQTGDS ESVPDPQPLG EPPAAPSSVG |
| 201 | SGTVAAGGGA PMADNNEGAD GVGNASGNWH CDSTWLGDRV ITTSTRTWAL |
| 251 | PTYNNHLYKQ ISSETAGSIN DNTYFGYSTP WGYFDFNRFH CHFSPRDWQR |
| 301 | LINNNWGFRP KKLRFKLFNI QVKEVTINDG VTTIANNLTS TIQVFSDSEY |
| 351 | QLPYVLGSAH QGCLPPFPAD VFMIPQYGYL TLNNGSQSVG RSSFYCLEYF |
| 401 | PSQMLRTGNN FEFSYSFEDV PFHSSYAHSQ SLDRLMNPLI DQYLYYLART |
| 451 | QSNPGGTAGN RELQFYQGGP SIMAEQAKNW LPGPCFRQQR VSKILDQNNN |
| 501 | SNFAWTGATK YHLNGRNSLV NPGVAMATHK DDEDRFFPSS GVLIFGKTGA |
| 551 | TNKTTLENVL MTNEEEIRPT NPVATEEYGI VSSNLQAANT AAQTQVVNNQ |
| 601 | GALPGMVWQN RDVYLQGPIW AKIPHTDGNF HPSPLMGGFG LKHPPPQILI |
| 651 | KNTPVPANPP EVFTPAKFAS FITQYSTGQV SVEIEWELQK ENSKRWNPEI |
| 701 | QYTSNFEKQT GVDFAVDSQG VYSEPRPIGT RYLTRNL |

### Example of Wild-Type AAV8 Capsid Protein Sequence

| (SEQ ID NO: 8) | |
|---|---|
| 1 | MAADGYLPDW LEDNLSEGIR EWWALKPGAP KPKANQQKQD DGRGLVLPGY |
| 51 | KYLGPFNGLD KGEPVNAADA AALEHDKAYD QQLQAGDNPY LRYNHADAEF |
| 101 | QERLQEDTSF GGNLGRAVFQ AKKRVLEPLG LVEEGAKTAP GKKRPVEPSP |
| 151 | QRSPDSSTGI GKKGQQPARK RLNFGQTGDS ESVPDPQPLG EPPAAPSGVG |
| 201 | PNTMAAGGGA PMADNNEGAD GVGSSSGNWH CDSTWLGDRV ITTSTRTWAL |
| 251 | PTYNNHLYKQ ISNGTSGGAT NDNTYFGYST PWGYFDFNRF HCHFSPRDWQ |
| 301 | RLINNNWGFR PKRLSFKLEN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRTGN NFQFTYTFED VPFHSSYAHS QSLDRLMNPL IDQYLYYLSR |
| 451 | TQTTGGTANT QTLGFSQGGP NTMANQAKNW LPGPCYRQQR VSTTTGQNNN |
| 501 | SNFAWTAGTK YHLNGRNSLA NPGIAMATHK DDEERFFPSN GILIFGKQNA |
| 551 | ARDNADYSDV MLTSEEEIKT TNPVATEEYG IVADNLQQQN TAPQIGTVNS |
| 601 | QGALPGMVWQ NRDVYLQGPI WAKIPHTDGN FHPSPLMGGF GLKHPPPQIL |
| 651 | IKNTPVPADP PTTFNQSKLN SFITQYSTGQ VSVEIEWELQ KENSKRWNPE |
| 701 | IQYTSNYYKS TSVDFAVNTE GVYSEPRPIG TRYLTRNL |

### Example of Wild-Type AAV9 Capsid Protein Sequence

| (SEQ ID NO: 9) | |
|---|---|
| 1 | MAADGYLPDW LEDNLSEGIR EWWALKPGAP QPKANQQHQD NARGLVLPGY |
| 51 | KYLGPGNGLD KGEPVNAADA AALEHDKAYD QQLKAGDNPY LKYNHADAEF |
| 101 | QERLKEDTSF GGNLGRAVFQ AKKRLLEPLG LVEEAAKTAP GKKRPVEQSP |
| 151 | QEPDSSAGIG KSGAQPAKKR LNFGQTGDTE SVPDPQPIGE PPAAPSGVGS |
| 201 | LTMASGGGAP VADNNEGADG VGSSSGNWHC DSQWLGDRVI TTSTRTWALP |
| 251 | TYNNHLYKQI SNSTSGGSSN DNAYFGYSTP WGYFDFNRFH CHFSPRDWQR |
| 301 | LINNNWGFRP KRLNFKLFNI QVKEVTDNNG VKTIANNLTS TVQVFTDSDY |
| 351 | QLPYVLGSAH EGCLPPFPAD VFMIPQYGYL TLNDGSQAVG RSSFYCLEYF |
| 401 | PSQMLRIGNN FQFSYEFENV PFHSSYAHSQ SLDRLMNPLI DQYLYYLSKT |
| 451 | INGSGQNQQT LKFSVAGPSN MAVQGRNYIP GPSYRQQRVS TTVTQNNNSE |
| 501 | FAWPGASSWA LNGRNSLMNP GPAMASHKEG EDRFFPLSGS LIFGKQGTGR |
| 551 | DNVDADKVMI TNEEEIKTTN PVATESYGQV ATNHQSAQAQ AQTGWVQNQG |
| 601 | ILPGMVWQDR DVYLQGPIWA KIPHTDGNFH PSPLMGGFGM KHPPPQILIK |
| 651 | NTPVPADPPT AFNKDKLNSF ITQYSTGQVS VEIEWELQKE NSKRWNPEIQ |
| 701 | YTSNYYKSNN VEFAVNTEGV YSEPRPIGTR YLTRNL |

### Example of Wild-Type AAV10 Capsid Protein Sequence

| (SEQ ID NO: 10) | |
|---|---|
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPVEPSP |
| 151 | QRSPDSSTGI GKKGQQPAKK RLNFGQTGDS ESVPDPQPIG EPPAGPSGLG |
| 201 | SGTMAAGGGA PMADNNEGAD GVGSSSGNWH CDSTWLGDRV ITTSTRTWAL |
| 251 | PTYNNHLYKQ ISNGTSGGST NDNTYFGYST PWGYFDFNRF HCHFSPRDWQ |
| 301 | RLINNNWGFR PKRLNFKLEN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRTGN NFEFSYQFED VPFHSSYAHS QSLDRLMNPL IDQYLYYLSR |
| 451 | TQSTGGTAGT QQLLFSQAGP NNMSAQAKNW LPGPCYRQQR VSTTLSQNNN |
| 501 | SNFAWTGATK YHLNGRDSLV NPGVAMATHK DDEERFFPSS GVLMFGKQGA |
| 551 | GKDNVDYSSV MLTSEEEIKT TNPVATEQYG VVADNLQQQN AAPIVGAVNS |
| 601 | QGALPGMVWQ NRDVYLQGPI WAKIPHTDGN FHPSPLMGGF GLKHPPPQIL |
| 651 | IKNTPVPADP PTTFSQAKLA SFITQYSTGQ VSVEIEWELQ KENSKRWNPE |
| 701 | IQYTSNYYKS INVDFAVNTD GTYSEPRPIG TRYLTRNL |

### Example of Wild-Type AAV11 Capsid Protein Sequence

| (SEQ ID NO: 11) | |
|---|---|
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPLESPQ |
| 151 | EPDSSSGIGK KGKQPARKRL NFEEDTGAGD GPPEGSDTSA MSSDIEMRAA |
| 201 | PGGNAVDAGQ GSDGVGNASG DWHCDSTWSE GKVTTTSTRT WVLPTYNNHL |
| 251 | YLRLGTTSSS NTYNGFSTPW GYFDFNRFHC HFSPRDWQRL INNNWGLRPK |
| 301 | AMRVKIFNIQ VKEVTTSNGE TTVANNLIST VQIFADSSYE LPYVMDAGQE |
| 351 | GSLPPFPNDV FMVPQYGYCG IVTGENQNQT DRNAFYCLEY FPSQMLRTGN |
| 401 | NFEMAYNFEK VPFHSMYAHS QSLDRLMNPL LDQYLWHLQS TTSGETLNQG |
| 451 | NAATTFGKIR SGDFAFYRKN WLPGPCVKQQ RFSKTASQNY KIPASGGNAL |
| 501 | LKYDTHYTLN NRWSNIAPGP PMATAGPSDG DFSNAQLIFP GPSVTGNTTT |
| 551 | SANNLLFTSE EEIAATNPRD TDMFGQIADN NQNATTAPIT GNVTAMGVLP |
| 601 | GMVWQNRDIY YQGPIWAKIP HADGHFHPSP LIGGFGLKHP PPQIFIKNTP |
| 651 | VPANPATTFT AARVDSFITQ YSTGQVAVQI EWEIEKERSK RWNPEVQFTS |
| 701 | NYGNQSSMLW APDTTGKYTE PRVIGSRYLT NHL |

### Example of Wild-Type AAV12 Capsid Protein Sequence

| (SEQ ID NO: 12) | |
|---|---|
| 1 | MAADGYLPDW LEDNLSEGIR EWWALKPGAP QPKANQQHQD NGRGLVLPGY |
| 51 | KYLGPFNGLD KGEPVNEADA AALEHDKAYD KQLEQGDNPY LKYNHADAEF |
| 101 | QQRLATDTSF GGNLGRAVFQ AKKRILEPLG LVEEGVKTAP GKKRPLEKTP |
| 151 | NRPTNPDSGK APAKKKQKDG EPADSARRTL DFEDSGAGDG PPEGSSSGEM |
| 201 | SHDAEMRAAP GGNAVEAGQG ADGVGNASGD WHCDSTWSEG RVTTTSTRTW |
| 251 | VLPTYNNHLY LRIGTTANSN TYNGFSTPWG YFDFNRFHCH FSPRDWQRLI |
| 301 | NNNWGLRPKS MRVKIFNIQV KEVTTSNGET TVANNLTSTV QIFADSTYEL |
| 351 | PYVMDAGQEG SFPPFPNDVF MVPQYGYCGV VTGKNQNQTD RNAFYCLEYF |
| 401 | PSQMLRTGNN FEVSYQFEKV PFHSMYAHSQ SLDRMMNPLL DQYLWHLQST |
| 451 | TTGNSLNQGT ATTTYGKITT GDFAYYRKNW LPGACIKQQK FSKNANQNYK |
| 501 | IPASGGDALL KYDTHTTLNG RWSNMAPGPP MATAGAGDSD FSNSQLIFAG |
| 551 | PNPSGNITTS SNNLLFTSEE EIATTNPRDT DMFGQIADNN QNATTAPHIA |
| 601 | NLDAMGIVPG MVWQNRDIYY QGPIWAKVPH TDGHFHPSPL MGGFGLKHPP |
| 651 | PQIFIKNTPV PANPNTTFSA ARINSFLTQY STGQVAVQID WEIQKEHSKR |
| 701 | WNPEVQFTSN YGTQNSMLWA PDNAGNYHEL RAIGSRFLTH HL |

### Example of Wild-Type AAV13 Capsid Protein Sequence

| (SEQ ID NO: 13) | |
|---|---|
| 1 | MTDGYLPDWL EDNLSEGVRE WWALQPGAPK PKANQQHQDN ARGLVLPGYK |
| 51 | YLGPGNGLDK GEPVNAADAA ALEHDKAYDQ QLKAGDNPYL KYNHADAEFQ |
| 101 | ERLQEDTSFG GNLGRAVFQA KKRILEPLGL VEEAAKTAPG KKRPVEQSPA |
| 151 | EPDSSSGIGK SGQQPARKRL NFGQTGDTES VPDPQPLGQP PAAPSGVGST |
| 201 | TMASGGGAPM ADNNEGADGV GNSSGNWHCD SQWLGDRVIT TSTRTWALPT |
| 251 | YNNHLYKQIS SQSGATNDNH YFGYSTPWGY FDFNRFHCHF SPRDWQRLIN |
| 301 | NNWGFRPKRL NFKLFNIQVK EVTQNDGTTT IANNLTSTVQ VFTDSEYQLP |
| 351 | YVLGSAHQGC LPPFPADVFM VPQYGYLTLN NGSQAVGRSS FYCLEYFPSQ |
| 401 | MLRTGNNFQF SYTFEDVPFH SSYAHSQSLD RLMNPLIDQY LYYLNRTQTA |
| 451 | SGTQQSRLLF SQAGPTSMSL QAKNWLPGPC YRQQRLSKQA NDNNNSNFPW |
| 501 | TGATKYHLNG RDSLVNPGPA MASHKDDKEK FFPMHGTLIF GKEGTNANNA |
| 551 | DLENVMITDE EEIRTTNPVA TEQYGTVSNN LQNSNAGPTT GTVNHQGALP |
| 601 | GMVWQDRDVY LQGPIWAKIP HTDGHFHPSP LMGGFGLKHP PPQIMIKNTP |
| 651 | VPANPPTNFS AAKFASFITQ YSTGQVSVEI EWELQKENSK RWNPEIQYTS |
| 701 | NYNKSVNVDF TVDINGVYSE PRPIGTRYLT RNL |

### Example of Wild-Type AAVrh10 Capsid Protein Sequence

| (SEQ ID NO: 14) | |
|---|---|
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPVEPSP |
| 151 | QRSPDSSTGI GKKGQQPAKK RLNFGQTGDS ESVPDPQPIG EPPAGPSGLG |
| 201 | SGTMAAGGGA PMADNNEGAD GVGSSSGNWH CDSTWLGDRV ITTSTRTWAL |
| 251 | PTYNNHLYKQ ISNGTSGGST NDNTYFGYST PWGYFDFNRF HCHFSPRDWQ |
| 301 | RLINNNWGFR PKRLNFKLEN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRIGN NFEFSYQFED VPFHSSYAHS QSLDRLMNPL IDQYLYYLSR |
| 451 | TQSTGGTAGT QQLLFSQAGP NNMSAQAKNW LPGPCYRQQR VSTTLSQNNN |
| 501 | SNFAWTGATK YHLNGRDSLV NPGVAMATHK DDEERFFPSS GVLMFGKQGA |
| 551 | GKDNVDYSSV MLTSEEEIKT TNPVATEQYG VVADNLQQQN AAPIVGAVNS |
| 601 | QGALPGMVWQ NRDVYLQGPI WAKIPHTDGN FHPSPLMGGF GLKHPPPQIL |
| 651 | IKNTPVPADP PTTFSQAKLA SFITQYSTGQ VSVEIEWELQ KENSKRWNPE |
| 701 | IQYTSNYYKS TNVDFAVNTD GTYSEPRPIG TRYLTRNL |

### Modified AAV Capsid Proteins

Provided herein are AAV capsid proteins having one or more modifications characterized by amino acid substitutions and/or peptide insertions. A modified AAV capsid protein may be modified from any wild-type capsid protein, for example an AAV1, AAV9, or AAVrh10 capsid protein. An AAV capsid protein comprising an amino acid substitution may comprise a substitution of a native amino acid (i.e., an amino acid found in a wild-type capsid protein) with a non-native amino acid (i.e., an amino acid not found at the given position in the corresponding wild-type capsid protein). An AAV capsid protein comprising a peptide insertion may comprise a non-native peptide (e.g., a non-native peptide provided herein) inserted between two amino acids of a wild-type AAV capsid protein, or may comprise a non-native peptide replacing one or more amino acid(s) of the corresponding wild-type AAV capsid protein. In instances in which a non-native peptide is inserted between two amino acids of a wild-type AAV capsid protein or replaces a segment of the capsid protein that is shorter than the non-native peptide, the resulting modified capsid protein is longer than the corresponding wild-type capsid protein, e.g., by 1, 2, 3, 4, 5, 6, 7, 8, 9, 10, 11, 12, 13, 14, 15, 16, 17, 18, 19, 20, 21, or more (up to the full length of the non-native peptide) amino acids. In some embodiments, the resulting modified capsid protein is 4 amino acids longer than the corresponding wild-type capsid protein. In some embodiments, the resulting modified capsid protein is 12 amino acids longer than the corresponding wild-type capsid protein.

In some embodiments, an AAV capsid protein disclosed herein comprises an amino acid substitution at a position corresponding to F535 of the wild-type AAV1 capsid protein of SEQ ID NO: 1. In some embodiments, an AAV capsid protein disclosed herein comprises an arginine (R) amino acid substitution at a position corresponding to F535 of the wild-type AAV1 capsid protein of SEQ ID NO: 1.

In some embodiments, an AAV capsid protein disclosed herein comprises a non-native peptide inserted at a particular site within the capsid protein or replacing particular amino acid(s) of the capsid protein. In some embodiments, the non-native peptide is a P2 peptide, such as a P2 peptide having an amino acid sequence which comprises, consists essentially of, or consists of the sequence GRILARGEINFK (SEQ ID NO: 15). In some embodiments, the non-native peptide is a C3 peptide, such as a C3 peptide having an amino acid sequence which comprises, consists essentially of, or consists of the sequence ASKKPKRNIKA (SEQ ID NO: 16). In some embodiments, the non-native peptide is an NBP10 peptide, such as an NBP10 peptide having an amino acid sequence which comprises, consists essentially of, or consists of the sequence AKKMWKKTW (SEQ ID NO: 17). In some embodiments, the non-native peptide is a P1B peptide, such as a P1B peptide having an amino acid sequence which comprises, consists essentially of, or consists of the sequence GEISVGESKFFL (SEQ ID NO: 18). In some embodiments, the non-native peptide is a P3DE peptide, such as a P3DE peptide having an amino acid sequence which comprises, consists essentially of, or consists of the sequence KHIFSDDSSELTIRNVDKNDE (SEQ ID NO: 19). In some embodiments, the non-native peptide is a P3G peptide, such as a P3G peptide having an amino acid sequence which comprises, consists essentially of, or consists of the sequence SIHLKVFAK (SEQ ID NO: 20).

In some embodiments, a non-native peptide (e.g., for insertion into a modified capsid protein) comprises, consists essentially of, or consists of an amino acid sequence having 60%, 65%, 70%, 75%, 80%, 85%, 90%, 91%, 92%, 93%, 94%, 95%, or more sequence identity with the sequence of any one of SEQ ID NOs: 15-20. In some embodiments, a non-native peptide (e.g., for insertion into a modified capsid protein) comprises, consists essentially of, or consists of an amino acid sequence having 1, 2, 3, 4, or 5 amino acid deletions relative to the sequence of any one of SEQ ID NOs: 15-20. In some embodiments, a non-native peptide (e.g., for insertion into a modified capsid protein) comprises, consists essentially of, or consists of an amino acid sequence having 1, 2, 3, 4, or 5 amino acid substitutions relative to the sequence of any one of SEQ ID NOs: 15-20. In some embodiments, a non-native peptide (e.g., for insertion into a modified capsid protein) comprises, consists essentially of, or consists of an amino acid sequence having 1, 2, 3, 4, or 5 additional amino acids relative to the sequence of any one of SEQ ID NOs: 15-20. In some embodiments, a non-native peptide comprises one or more modifications relative to the sequence of any one of SEQ ID NOs: 15-20, wherein the modifications are selected from an amino acid deletion, an amino acid substitution, and an additional amino acid.

In some embodiments, a non-native peptide is inserted into or replaces amino acids of a surface variable region (VR) loop of an AAV capsid protein disclosed herein. The VRs of AAV capsid proteins are evolutionarily divergent sequences within capsid proteins which are localized at the surface of an assembled viral capsid, and are considered to be responsible for much or all interaction with cell surface receptors and other host factors (see, e.g., Govindasamy, et al., J. Virol 2006 80(23):11556-11570; Govindasamy, et al., J. Virol. 2013 87(20):11187-11191; and DiMattia, et al, J. Virol. 2012 86(12):6947-6958). In some embodiments, the non-native peptide is inserted into or replaces amino acid(s) of VR loop I (VR-I), VR-II, VR-III, VR-IV, VR-V, VR-VI, VR-VII, VR-VIII, or VR-IX. Each of the VR loops (VR-I through VR-IX) are present within the VP3 capsid protein. As such, they are also present within the longer VP1 and VP2 capsid proteins. In some embodiments, the non-native peptide is inserted into or replaces amino acid(s) of VR-IV or VR-VIII. In some embodiments, the non-native peptide is inserted into or replaces amino acid(s) of VR-IV or VR-VIII of an AAV1, AAV9, or AAVrh10 capsid protein. Exemplary positions of the VR loops, according to the numbering of the wild-type AAV1, AAV9, and AAVrh10 VP1 capsid proteins (provided by SEQ ID NOs: 1, 9, and 14, respectively) are provided in Table 1 below.

TABLE 1
Examples of AAV capsid protein variable regions
and their corresponding amino acid positions*

| VR Loop | AAV1 positions | AAV9 positions | AAVrh10 positions |
|---|---|---|---|
| VR-I | 261-268 | 261-269 | 262-270 |
| VR-II | 326-331 | 327-332 | 328-333 |
| VR-III | 381-385 | 382-386 | 383-389 |
| VR-IV | 451-459 | 452-459 | 453-461 |
| VR-V | 488-505 | 488-505 | 490-507 |
| VR-VI | 527-539 | 527-539 | 529-541 |
| VR-VII | 545-558 | 545-558 | 547-560 |
| VR-VIII | 581-593 | 581-593 | 583-595 |
| VR-IX | 704-714 | 704-714 | 706-716 |

*Amino acid positions in Table 1 are provided according to the full-length VP1 capsid protein numbering (e.g., according to the sequences of SEQ ID NOs: 1, 9, and 14).

In some embodiments, a non-native peptide replaces one or more (e.g., 1, 2, 3, 4, 5, 6, 7, 8, 9, or more) amino acids of a VR loop of a capsid protein disclosed herein. In some embodiments, a non-native peptide replaces one or more (e.g., 1, 2, 3, 4, 5, 6, 7, 8, or 9) amino acids of VR-IV. In some embodiments, a non-native peptide replaces amino acid residues of VR-IV corresponding to amino acids 451-458 of the wild-type AAV1 capsid protein of SEQ ID NO: 1. In some embodiments, a non-native peptide replaces one or more (e.g., 1, 2, 3, 4, 5, 6, 7, 8, 9, 10, 11, 12, or 13) amino acids of VR-VIII.

In some embodiments, a non-native peptide is inserted between two amino acids of a VR loop of a capsid protein disclosed herein. In some embodiments, a non-native peptide is inserted in VR-IV between amino acids corresponding to positions 450 and 451, 451 and 452, 452 and 453, 453 and 454, 454 and 455, 455 and 456, 456 and 457, 457 and 458, or 458 and 459 of the wild-type AAV1 capsid protein of SEQ ID NO: 1. In some embodiments, a non-native peptide is inserted in VR-VIII between amino acids corresponding to positions 581 and 582, 582 and 583, 583 and 584, 584 and 585, 585 and 586, 586 and 587, 587 and 588, 588 and 589, 589 and 590, 590 and 591, 591 and 592, or 592 and 593 of the wild-type AAV1 capsid protein of SEQ ID NO: 1. In some embodiments, a non-native peptide is inserted in VR-VIII between amino acids corresponding to S587 and S588 of the wild-type AAV1 capsid protein of SEQ ID NO: 1.

In some embodiments, an AAV capsid protein disclosed herein comprises a combination of two or more modifications disclosed herein (e.g., an amino acid substitution and a non-native peptide insertion or replacement, a non-native peptide insertion or replacement at more than one site within the capsid protein, or more than one distinct non-native peptides inserted within the capsid protein or replacing amino acid(s) of the capsid protein).

In some embodiments, an AAV capsid protein that comprises a modification (e.g., an amino acid substitution or a non-native peptide insertion) does not comprise additional modifications. An AAV capsid protein that does not comprise an additional modification, therefore, has an amino acid sequence that is identical to the amino acid sequence of the corresponding wild-type capsid protein except for the modification. For example, a modified AAV capsid protein may in some embodiments have an amino acid sequence that is identical to the amino acid sequence of a wild-type capsid protein except for at the locus at which a non-native peptide is inserted, or at which a non-native amino acid is substituted. As such, a modified AAV capsid protein may comprise an amino acid sequence that has 80%, 85%, 90%, 91%, 92%, 93%, 94%, 95%, 96%, 97%, 98%, or 99% identity with the amino acid sequence of a corresponding wild-type AAV capsid protein. In some embodiments, a modified AAV capsid protein comprises 1, 2, 3, 4, 5, 6, 7, 8, 9, 10, 11, 12, 13, 14, 15, 16, 17, 18, 19, 20, or 21 amino acid residues (e.g., the length of a non-native peptide that is inserted into the modified capsid protein) that are not present in the amino acid sequence of the corresponding wild-type capsid protein.

Also provided herein are nucleic acids encoding capsid proteins (e.g., modified capsid proteins). A nucleic acid may comprise, consist essentially of, or consist of a sequence that encodes a capsid protein disclosed here (e.g., a capsid protein comprising one or more amino acid substitutions and/or peptide insertions). A sequence encoding a capsid protein disclosed herein can be determined by one of ordinary skill in the art by known methods. A nucleic acid encoding a capsid protein may comprise a promoter or other regulatory sequence operably linked to the coding sequence. A nucleic acid encoding a capsid protein may be in the form of a plasmid, an mRNA, or another nucleic acid capable of being used by enzymes or machinery of a host cell to produce a capsid protein. Nucleic acids encoding capsid proteins as provided herein can be used to make AAV particles that can be used, e.g., for delivering a therapeutic or diagnostic agent to a cell. Methods of making AAV particles are known in the art. For example, see Scientific Reports volume 9, Article number: 13601 (2019); Methods Mol Biol. 2012; 798: 267-284; and www.thermofisher.com/us/en/home/clinical/cell-gene-therapy/gene-therapy/aav-production-workflow.html.

### Modified AAV Particles

According to some aspects, AAV particles are provided herein. In some embodiments, an AAV particle comprises, consists essentially of, or consists of an empty capsid (e.g., a capsid without a cargo). In some embodiments, an AAV particle comprises, consists essentially of, or consists of a capsid encapsidating a nucleic acid (e.g., a nucleic acid vector that comprises a gene of interest). In some embodiments, a nucleic acid encapsidated within an AAV capsid to generate an AAV particle comprises, consists essentially of, or consists of a nucleic acid vector disclosed herein. In some embodiments, an AAV particle disclosed herein comprises, consists essentially of, or consists of a capsid protein comprising one or more modifications disclosed herein, such as modifications characterized by amino acid substitutions and/or peptide insertions.

In some embodiments, an AAV particle disclosed herein is replicative. A replicative AAV particle is capable of replicating within a host cell (e.g., a host cell within a subject or a host cell in culture). In some embodiments, an AAV particle disclosed herein is non-replicating. A non-replicating AAV particle is not capable of replicating within a host cell (e.g., a host cell within a subject or a host cell in culture), but can infect the host and incorporate genetic components into the host's genome for expression. In some embodiments, an AAV particle disclosed herein is capable of infecting a host cell. In some embodiments, an AAV particle disclosed herein is capable of facilitating stable integration of genetic components into the genome of a host cell. In some embodiments, an AAV particle disclosed herein is not capable of facilitating integration of genetic components into the genome of a host cell.

In some embodiments, an AAV particle disclosed herein comprises a nucleic acid vector. In some embodiments, a nucleic acid vector comprises, consists essentially of, or consists of two inverted terminal repeats (ITRs) adjacent to the ends of a sequence encoding a gene of interest. In some embodiments, the nucleic acid vector is comprised within the AAV's ssDNA genome. In some embodiments, an AAV particle disclosed herein comprises one single-stranded DNA. In some embodiments, an AAV particle disclosed herein comprises two complementary DNA strands, forming a self-complementary AAV (scAAV).

An AAV particle disclosed herein may be of any AAV serotype (e.g., AAV serotype 1, 2, 3, 4, 5, 6, 7, 8, 9, 10, 11, 12, or 13), including any derivative (including non-naturally occurring variants of a serotype) or pseudotype. Non-limiting examples of derivatives and pseudotypes include AAV2-AAV3 hybrid, AAVrh10, AAVhu.14, AAV3a/3b, AAVrh32.33, AAV-HSC15, AAV-HSC17, AAVhu.37, AAVrh8, CHt-P6, AAV2.5, AAV6.2, AAV2i8, AAV-HSC15/17, AAVM41, AAV9.45, AAV2.5T, AAV-HAE1/2, AAV clone 32/83, AAVShH10, AAV2.15, AAV2.4, AAVM41, and AAVr3.45. Such AAV serotypes and derivatives/pseudotypes, and methods of producing such derivatives/pseudotypes are known in the art (see, e.g., Asokan, et al., Mol. Ther. 2012 April; 20(4):699-708. doi: 10.1038/mt.2011.287). In some embodiments, the AAV particle is a pseudotyped AAV particle, which comprises a nucleic acid vector comprising ITRs from one serotype and a capsid comprised of capsid proteins derived from another serotype. Methods for producing and using pseudotyped rAAV vectors are known in the art (see, e.g., Duan et al., J. Virol., 75:7662-7671 (2001); Halbert et al., J. Virol., 74:1524-1532 (2000); Zolotukhin et al., Methods, 28:158-167 (2002); and Auricchio et al., Hum. Molec. Genet., 10:3075-3081 (2001)). In some embodiments in which an AAV particle comprises both wild-type and modified capsid proteins, both the wild-type and modified capsid proteins are expressed in the same cell used to produce the AAV particle.

In some embodiments, an AAV particle disclosed herein is a recombinant AAV (rAAV) particle, e.g., comprising a recombinant nucleic acid or transgene.

In some embodiments, an AAV particle disclosed herein comprises one or more modified capsid proteins disclosed herein. In some embodiments, each capsid protein (e.g., each VP1 capsid protein, each VP2 capsid protein, each VP3 capsid protein, or each of two or more of VP1, VP2, and VP3 capsid proteins) of an AAV particle disclosed herein comprises the same modification (e.g., an amino acid substitution and/or peptide insertion). In some embodiments, a subset of the capsid proteins of an AAV particle disclosed herein do not comprise a modification (e.g., have the amino acid sequence of a wild-type AAV capsid protein).

In some embodiments, an AAV particle disclosed herein comprises both modified and unmodified (e.g., having a wild-type amino acid sequence) capsid proteins in a particular ratio. For example, in some embodiments, an AAV particle disclosed herein comprises wild-type and modified capsid proteins at a ratio of 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, or 5:95. In some embodiments, 5-95%, 10-95%, 15-95%, 20-95%, 25-95%, 30-95%, 35-95%, 40-95%, 45-95%, 50-95%, 55-95%, 60-95%, 65-95%, 70-95%, 75-95%, 80-95%, 85-95%, 90-95%, 5-90%, 10-90%, 15-90%, 20-90%, 25-90%, 30-90%, 35-90%, 40-90%, 45-90%, 50-90%, 55-90%, 60-90%, 65-90%, 70-90%, 75-90%, 80-90%, 85-90%, 5-80%, 10-80%, 15-80%, 20-80%, 25-80%, 30-80%, 35-80%, 40-80%, 45-80%, 50-80%, 55-80%, 60-80%, 65-80%, 70-80%, 75-80%, 5-70%, 10-70%, 15-70%, 20-70%, 25-70%, 30-70%, 35-70%, 40-70%, 45-70%, 50-70%, 55-70%, 60-70%, 65-70%, 5-60%, 10-60%, 15-60%, 20-60%, 25-60%, 30-60%, 35-60%, 40-60%, 45-60%, 50-60%, 55-60%, 5-50%, 10-50%, 15-50%, 20-50%, 25-50%, 30-50%, 35-50%, 40-50%, or 45-50% of the capsid proteins comprised within an AAV particle disclosed herein are modified capsid proteins. In some embodiments, 50% or about 50% of the capsid proteins comprised within an AAV particle disclosed herein are modified capsid proteins. In some embodiments, 65% or about 65% of the capsid proteins comprised within an AAV particle disclosed herein are modified capsid proteins. In some embodiments, at least 50% of the capsid proteins comprised within an AAV particle disclosed herein are modified capsid proteins. In some embodiments, the modified capsid protein comprises a non-native peptide insertion or substitution in VR-IV or VR-VIII, and the unmodified capsid protein has the amino acid sequence of the corresponding wild-type AAV capsid protein. For example, in some embodiments, the modified capsid protein is an AAV1 capsid protein with a non-native peptide inserted or substituted within VR-IV and the wild-type capsid protein is a capsid protein having the amino acid sequence of a wild-type AAV1 capsid protein.

In some embodiments, VP1 proteins, VP2 proteins, and VP3 proteins each comprise modifications. In some embodiments in which an AAV particle comprises unmodified and modified capsid proteins, the VP1 proteins, VP2 proteins, and VP3 proteins of the AAV particle comprise the same ratio of unmodified:modified, or the same or approximately the same percentage of VP1 proteins, VP2 proteins, and VP3 proteins are modified. In some embodiments, only VP1 proteins are modified, only VP2 proteins are modified, or only VP3 proteins are modified. In some embodiments, only VP1 and VP2 proteins are modified, only VP2 and VP3 proteins are modified, or only VP1 and VP3 proteins are modified. In some embodiments, VP1, VP2 and VP3 proteins are modified.

### AAV Targeting

Some aspects of the present disclosure provide modified AAV capsid proteins and/or AAV particles comprising modified capsid proteins which have enhanced binding properties relative to corresponding wild-type AAV capsid proteins/particles to particular proteins, cells, or tissues of interest, and/or have enhanced targeting to (e.g., resulting in enhanced accumulation within) particular cells or tissues of a subject. In some embodiments, a modified capsid protein has enhanced binding to a biological molecule (e.g., a polypeptide or a polysaccharide, such as a polypeptide or polysaccharide on or comprised within a cell or tissue) relative to a corresponding wild-type (unmodified) capsid protein. In some embodiments, a modified capsid protein has enhanced binding to a glycan, such as a glycan of a glycoprotein, glycolipid, or proteoglycan, relative to a corresponding wild-type (unmodified) capsid protein. In some embodiments, a modified capsid protein has enhanced binding to a sialoglycan (a glycan comprising sialic acid) relative to a corresponding wild-type (unmodified) capsid protein. In some embodiments, a modified capsid protein has enhanced binding to a glycan (e.g., a sialoglycan) attached to a polypeptide relative to a corresponding wild-type (unmodified) capsid protein. In some embodiments, a modified capsid protein has enhanced binding to a glycan (e.g., a sialoglycan) of a neural cell adhesion molecule relative to a corresponding wild-type (unmodified) capsid protein. It should be understood that a modified capsid protein having enhanced binding to a biological molecule can confer such enhanced binding properties to an AAV particle comprising the modified capsid protein. Enhanced binding to a biological molecule can confer enhanced targeting to particular proteins, cells, or tissues of interest (e.g., for selective delivery of an AAV particle and/or therapeutic/diagnostic molecule therein or encoded therefrom to said protein, cell, or tissue of interest). In some embodiments, enhanced binding properties may correspond to a decrease in binding to a particular protein, cell, or tissue, e.g., such that a modified capsid protein or AAV particle comprising a modified capsid protein bind to or associate with said protein, cell, or tissue to a lesser extent than a corresponding wild-type (unmodified) capsid protein or an AAV particle not comprising a modified capsid protein. A decrease in binding and/or association with a particular protein, cell, or tissue may have the beneficial effect of decreasing accumulation of an AAV particle comprising a modified capsid protein within an undesired cell type and/or within an undesired tissue.

As used herein, binding or binding properties refer to the strength and/or specificity of interaction between an AAV capsid protein (e.g., a modified AAV capsid protein) or AAV particle and a particular target (e.g., a protein, cell, or tissue). In some embodiments enhanced binding refers to an increase (e.g., for a desired target compound) or decrease (e.g., for an undesired off-target compound) in the affinity of the interaction between an AAV capsid protein (e.g., a modified AAV capsid protein) and a particular compound (e.g., a protein or glycan of interest, such as on a cell or in a tissue). The affinity of an interaction can be measured using techniques known in the art (see, e.g., Jarmoskaite, et al., eLife 2020 9:e57264 doi: 10.7554/eLife.57264). In some embodiments, the affinity of an interaction between a modified AAV capsid protein and a compound (e.g., a protein or glycan of interest) is characterized by an equilibrium dissociation constant (K_{D}). In some embodiments, the K_{D} is less than 10⁻⁴ M, less than 10⁻⁵ M, less than 10⁻⁶ M, less than 10⁻⁷ M, less than 10⁻⁸ M, less than 10⁻⁹ M, less than 10⁻¹⁰ M, less than 10⁻¹¹ M, or less than 10⁻¹² M.

In some embodiments, the affinity of an interaction between a modified AAV capsid protein and a compound (e.g., a protein or glycan of interest) is increased for the modified AAV capsid protein relative to a corresponding wild-type (e.g., unmodified) AAV capsid protein. In some such embodiments, the K_{D} for the interaction between the modified AAV capsid protein and the compound is 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1000-fold, 2000-fold, 3000-fold, 4000-fold, 5000-fold, 10⁴-fold, or more, lower than the K_{D} for the interaction between a corresponding wild-type (e.g., unmodified) AAV capsid protein and the compound.

In some embodiments, the affinity of an interaction between a modified AAV capsid protein and a compound (e.g., a protein or glycan of interest) is decreased for the modified AAV capsid protein relative to a corresponding wild-type (e.g., unmodified) AAV capsid protein. In some such embodiments, the K_{D} for the interaction between the modified AAV capsid protein and the compound is 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1000-fold, 2000-fold, 3000-fold, 4000-fold, 5000-fold, 10⁴-fold, or more, higher than the K_{D} for the interaction between a corresponding wild-type (e.g., unmodified) AAV capsid protein and the compound.

In some embodiments enhanced binding refers to an increase (e.g., for a desired target compound) or a decrease (e.g., for an undesired off-target compound) in the avidity of the interaction between an AAV capsid protein (e.g., a modified AAV capsid protein) or an AAV particle comprising a modified capsid protein and a particular compound (e.g., a protein or glycan cell, or tissue of interest). Avidity defines the total intermolecular force between multiple parallel interactions, such as between multiple modified capsid proteins on an AAV particle and multiple biomolecules on a cell or tissue of interest, and is often characterized for compounds having multivalent presentation of interaction moieties. Avidity of an interaction depends on the binding affinity of the interaction as well as the valency and structural arrangement of the interaction moieties.

In some embodiments, the avidity of an interaction between an AAV particle comprising a modified capsid protein and a target (e.g., a cell or tissue of interest) is increased for the particle comprising the modified capsid protein relative to a corresponding wild-type AAV particle (e.g., not comprising the modified capsid protein). In some such embodiments, the avidity for the interaction between the AAV particle comprising the modified capsid protein and the target is 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1000-fold, 2000-fold, 3000-fold, 4000-fold, 5000-fold, 10⁴-fold, or more, higher than the avidity for the interaction between a corresponding wild-type AAV particle (e.g., not comprising the modified capsid protein) and the target.

In some embodiments, the avidity of an interaction between an AAV particle comprising a modified AAV capsid protein and a target (e.g., a cell or tissue of interest) is decreased for the particle comprising the modified AAV capsid protein relative to a corresponding wild-type AAV particle (e.g., not comprising the modified capsid protein). In some such embodiments, the avidity for the interaction between the modified AAV capsid protein and the compound is 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1000-fold, 2000-fold, 3000-fold, 4000-fold, 5000-fold, 10⁴-fold, or more, lower than the avidity for the interaction between a corresponding wild-type AAV particle (e.g., not comprising the modified capsid protein) and the target.

In some embodiments, a modified AAV capsid protein has enhanced binding to a particular cell or tissue type of interest, relative to a corresponding wild-type (unmodified) capsid protein. In some embodiments, a modified AAV capsid protein has enhanced binding to a cell of the central and/or peripheral nervous system. In some embodiments, a modified AAV capsid protein has enhanced binding to neurons and/or glial cells. In some embodiments, a modified AAV capsid protein has enhanced binding to brain neurons. In some embodiments, a modified AAV capsid protein has enhanced binding to peripheral neurons. In some embodiments, a modified AAV capsid protein has enhanced binding to astrocytes, oligodendrocytes, microglia, and/or ependymal cells. In some embodiments, a modified AAV capsid protein has enhanced binding to Schwann cells and/or satellite cells.

In some embodiments, an AAV particle comprising a modified capsid protein provided herein accumulates within a tissue of interest following contacting the tissue to a greater extent than does a corresponding AAV particle that does not comprise the modified capsid protein. In some embodiments, an AAV particle comprising a modified capsid protein accumulates within a tissue of interest (e.g., within the brain and/or the spinal cord) following administration to a subject (e.g., by parenteral injection, such as intravenous injection or intrathecal injection) to a greater extent than does a corresponding AAV particle that does not comprise the modified capsid protein.

### Nucleic Acid Vectors

According to some aspects, provided herein are nucleic acid vectors that may be encapsidated by any one of the modified AAV capsids as provided herein. In some embodiments, a nucleic acid vector as provided herein comprises a first inverted terminal repeat (ITR) and a second ITR. A nucleic acid vector may comprise one or more heterologous nucleic acid sequences encoding a gene of interest (e.g., a protein or polypeptide of interest) and one or more sequences comprising inverted terminal repeat (ITR) sequences flanking the one or more heterologous nucleic acid sequences. In some embodiments, a nucleic acid vector is encapsidated within an AAV capsid forming an AAV particle. In some embodiments, a nucleic acid vector disclosed herein is encapsidated by an AAV capsid comprising a modified capsid protein, such as an AAV capsid comprising an amino acid substitution and/or a peptide insertion.

In some embodiments, a nucleic acid vector comprises native AAV genes or native AAV nucleotide sequences. In some embodiments, one or more native AAV genes or native AAV nucleotide sequences may be removed from a nucleic acid vector. In some embodiments, one or more native AAV genes or native AAV nucleotide sequences may be removed from a nucleic acid vector and replaced with a gene of interest.

A nucleic acid vector can be of any AAV serotype, such as AAV1, AAV2, AAV3, AAV4, AAV5, AAV6, AAV7, AAV8, AAV9, AAV10, AAV11, AAV12, AAV13, or AAVrh10, or a combination of serotypes. In some embodiments, a nucleic acid vector encapsidated within an AAV capsid forms a pseudotyped AAV particle, such that the nucleic acid vector is of a serotype distinct from the AAV capsid in which it is encapsidated. For example, a nucleic acid vector of serotype AAV2 may be encapsidated within a capsid of serotype AAVrh10.

In some embodiments, a nucleic acid vector is single-stranded and comprises a first inverted terminal repeat (ITR) and a second ITR. As disclosed herein, the first ITR refers to the ITR at the 5′ terminus of the nucleic acid vector, and the second ITR refers to the ITR at the 3′ terminus of the nucleic acid vector. Each ITR in its native or wild-type form is or is about 145 nucleotides in length (e.g., about 140 nucleotides, about 145 nucleotides, about 150 nucleotides, about 155 nucleotides, about 160 nucleotides, or about 165 nucleotides) and comprises a D-sequence. Each ITR can independently be of any AAV serotype (e.g., AAV1, AAV2, AAV3, AAV4, AAV5, AAV6, AAV7, AAV8, AAV9, AAV10, AAV11, AAV12, AAV13, or AAVrh10), or both ITRs may be of the same serotype. ITRs are described, for example, in Grimm et al. J. Virol. 80(1):426-439 (2006).

A nucleic acid vector as disclosed herein in some embodiments comprises one or more regulatory elements. A regulatory element refers to a nucleotide sequence or structural component of a nucleic acid vector which is involved in the regulation of expression of components of the nucleic acid vector (e.g., a gene of interest comprised therein). Regulatory elements include, but are not limited to, promoters, enhancers, silencers, insulators, response elements, initiation sites, termination signals, and ribosome binding sites.

Promoters include constitutive promoters, inducible promoters, tissue-specific promoters, cell type-specific promoters, and synthetic promoters. For example, a nucleic acid vector disclosed herein may include viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter.

Inducible promoters or other inducible regulatory elements may also be used to achieve desired expression levels of a gene of interest (e.g., a protein or polypeptide of interest). Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters or other tissue-specific regulatory elements are also contemplated herein. Non-limiting examples of such promoters that may be used include neuron-specific promoters, such as glutamic acid decarboxylase 65 (GAD65) promoter, synapsin (SYN) promoter, glial fibrillary acidic protein (GFAP) promoter, α-internexin (INA) promoter, nestin (NES) promoter, myelin associated oligodendrocyte basic protein (MOBP) promoter, myelin basic protein (MBP) promoter, tyrosine hydroxylase (TH) promoter, or forkhead box protein A2 (FOXA2/HNF30) promoter. See, for example, Hoshino et al., Mol. Brain 2021 14:33 doi: 10.1186/s13041-021-00746-1; Kugler et al., Gene Therapy 2003 10:337-347 doi: 10.1038/sj.gt.3301905; Miura & Mikoshiba, J. Neurochem. 1990 55(4):1180-1188 doi: 10.1111/j.1471-4159.1990.tb03123.x; Brenner et al., J. Neurosci. 1994 14(3 Pt 1):1030-1037 doi: 10.1523/JNEUROSCI.14-03-01030.1994; Ching & Liem, J. Biol. Chem. 1991 266(29):19459-19468; Beech et al., J. Comp. Neurol. 2004 475(1):128-141 doi: 10.1002/cne.20179; Wrabetz et al., J. Neurosci. Res. 1993 36(4):455-471 doi: 10.1002/jnr.490360412; Kessler et al., Brain Res. Mol. Brain Res. 2003 112(1-2):8-23 doi: 10.1016/s0169-328x(02)00694-0; and Rolland et al., Mol. Ther. Methods Clin. Dev. 2016 3:16062 doi: 10.1038/mtm.2016.62.

Synthetic promoters are also contemplated herein. A synthetic promoter may comprise, for example, regions of known promoters, regulatory elements, transcription factor binding sites, enhancer elements, repressor elements, and the like.

In some embodiments, a nucleic acid provided herein comprises a nucleotide sequence encoding a product (e.g., a protein or polypeptide product). In some embodiments, a nucleotide sequence comprises a nucleotide sequence of a gene of interest. In some embodiments, a gene of interest encodes a therapeutic or diagnostic protein or polypeptide. In some embodiments, a therapeutic or diagnostic protein or polypeptide is an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic agent, an enzyme, a bone morphogenetic protein, a nuclease, a protein used for gene editing, an Fc-fusion protein, an anticoagulant, or a protein or polypeptide that can be detected using a laboratory test. In some embodiments, a gene of interest encodes a protein having activity within the central nervous system (e.g., within a cell of the CNS and/or between cells of the CNS), such as a neurotransmitter receptor, a neuropeptide, or a membrane transport protein. In some embodiments, a gene of interest encodes alpha-L-iduronidase (IDUA), iduronate 2-sulfatase (IDS), N-sulfoglucosamine sulfohydrolase (SGSH), alpha-N-acetylglucosaminidase (NAGLU), tripeptidyl peptidase-I (TPP1/CLN2), CLN3 lysosomal/endosomal transmembrane protein, Battenin (CLN3), ceroid-lipofuscinosis neuronal protein 6 (CLN6), arylsulfatase A (ARSA), aspartoacylase (ASPA), aromatic L-amino acid decarboxylase (AADC), neurturin (NTN), glial cell derived neurotrophic factor (GDNF), nerve growth factor (NGF), apolipoprotein E2 (APOE2), or survival motor neuron (SMN). In some embodiments, a nucleic acid provided herein comprises a nucleotide sequence encoding a guide RNA or other nucleic acid used for gene editing, optionally in addition to a protein used for gene editing. Additional information regarding AAV delivery of genes of interest to the nervous system can be found, for example, in Hudry & Vandenberghe, Neuron 2019 101(5):839-862 doi: 10.1016/j.neuron.2019.03.020.

In some embodiments, a product encoded by a nucleic acid disclosed herein is a detectable molecule. A detectable molecule is a molecule that can be visualized (e.g., using a naked eye, under a microscope, or using a light detection device such as a camera). In some embodiments, the detectable molecule is a fluorescent molecule, a bioluminescent molecule, or a molecule that provides color (e.g., β-galactosidase, β-lactamase, β-glucuronidase, or spheroidenone). In some embodiments, the detectable molecule is a fluorescent, bioluminescent or enzymatic protein or functional peptide or polypeptide thereof.

In some embodiments, fluorescent protein is a blue fluorescent protein, a cyan fluorescent protein, a green fluorescent protein, a yellow fluorescent protein, an orange fluorescent protein, a red fluorescent protein, or a functional peptide or polypeptide thereof. A blue fluorescent protein may be azurite, EBFP, EBFP2, mTagBFP, or Y66H. A cyan fluorescent protein may be ECFP, AmCyanl, Cerulean, CyPet, mECFP, Midori-ishi Cyan, mTFP1, or TagCFP. A Green fluorescent protein may be AcGFP, Azami Green, EGFP, Emarald, GFP or a mutated form of GFP (e.g., GFP-S65T, mWasabi, Stemmer, Superfolder GFP, TagGFP, TurboGFP, or ZsGreen). A yellow fluorescent protein may be EYFP, mBanana, mCitrine, PhiYFp, TagYFP, Topaz, Venus, YPet, or ZsYellow1. An orange fluorescent protein may be DsRed, RFP, DsRed2, DsRed-Express, Ds-Red-monomer, Tomato, tdTomato, Kusabira Orange, mKO2, mOrange, mOrange2, mTangerine, TagRFP, or TagRFP-T. A red fluorescent protein may be AQ142, AsRed2, dKeima-Tandem, HcRed1, tHcRed, Jred, mApple, mCherry, mPlum, mRasberry, mRFP1, mRuby or mStrawberry.

In some embodiments, a detectable molecule is a bioluminescent protein or a functional peptide or polypeptide thereof. Non-limiting examples of bioluminescent proteins are firefly luciferase, click-beetle luciferase, Renilla luciferase, and luciferase from Oplophorus gracilirostris.

In some embodiments, a detectable molecule may be any polypeptide or protein that can be detected using methods known in the art. Non-limiting methods of detection are fluorescence imaging, luminescent imaging, bright filed imaging, and include imaging facilitated by immunofluorescence or immunohistochemical staining.

Additional features of AAV particles, nucleic acid vectors, and capsid proteins are described in U.S. Patent Publication No. 2017/0356009, the contents of which are incorporated herein by reference in their entirety.

### Immunogenicity of AAV

According to some aspects, modified AAV capsid proteins comprising one or more modifications provided herein have altered immunogenicity relative to unmodified AAV capsid proteins. In some embodiments, a modified AAV capsid protein is less immunogenic than a corresponding wild-type (e.g., unmodified) AAV capsid protein. In some embodiments, a modified AAV capsid protein is less susceptible to binding by neutralizing antibodies. In some embodiments, a modified AAV capsid protein is less susceptible to binding by ADKla, ADK1b, ADK9, HL2370, HL2374, ADK8, HL2381, and/or HL2383 antibodies. In some embodiments, a modified AAV capsid protein is less susceptible to recognition by an immune cell (e.g., a B cell or a T cell). In some embodiments, a modified AAV capsid protein is less susceptible to opsonization by neutralizing antibodies. It should be understood that the features (e.g., altered immunogenicity) of a modified AAV capsid protein may also be characteristic of an AAV particle comprising the modified AAV capsid protein. For example, in some embodiments, an AAV particle comprising a modified AAV capsid protein is less susceptible to binding by neutralizing antibodies than is a corresponding AAV particle that does not comprise the modified AAV capsid protein.

### Transduction Efficiency

According to some aspects, transduction efficiency of an AAV particle comprising a modified AAV capsid protein disclosed herein is altered relative to an AAV particle that does not comprise the modified AAV capsid protein. Transduction efficiency of an AAV particle can be determined, for example, by comparing expression of a gene of interest in a cell following contacting the cell with the AAV particle. In some embodiments, transduction efficiency of an AAV particle comprising a modified AAV capsid protein as disclosed herein is higher than the transduction efficiency of an AAV particle that does not comprise the modified capsid protein. In some embodiments, the transduction efficiency of an AAV particle comprising a modified AAV capsid protein as disclosed herein is at least 5% higher (e.g., at least 10% higher, at least 15% higher, at least 20% higher, at least 25% higher, at least 30% higher, at least 35% higher, at least 40% higher, at least 50% higher, at least 60% higher, at least 70% higher, at least 80% higher, at least 90% higher, at least 100% higher, at least 150% higher, at least 200% higher, at least 250% higher, or more) than the transduction efficiency of an AAV particle that does not comprise the modified capsid protein. In some embodiments, the transduction efficiency of an AAV particle comprising a modified AAV capsid protein as disclosed herein is at least 1.5-fold higher (e.g., at least 2-fold higher, at least 2.5-fold higher, at least 3-fold higher, at least 3.5-fold higher, at least 4-fold higher, at least 4.5-fold higher, at least 5-fold higher, at least 5.5-fold higher, at least 6-fold higher, at least 6.5-fold higher, at least 7-fold higher, at least 7.5-fold higher, at least 8-fold higher, at least 8.5-fold higher, at least 9-fold higher, at least 9.5-fold higher, at least 10-fold higher, at least 10.5-fold higher, at least 11-fold higher, at least 11.5-fold higher, at least 12-fold higher, at least 12.5-fold higher, at least 13-fold higher, at least 13.5-fold higher, at least 14-fold higher, at least 14.5-fold higher, at least 15-fold higher, at least 15.5-fold higher, at least 16-fold higher, at least 16.5-fold higher, at least 17-fold higher, at least 17.5-fold higher, at least 18-fold higher, at least 18.5-fold higher, at least 19-fold higher, at least 19.5-fold higher, at least 20-fold higher, or more) than the transduction efficiency of an AAV particle that does not comprise the modified capsid protein. In some embodiments, transduction efficiency of an AAV particle comprising a modified AAV capsid protein as disclosed herein is not altered relative to an AAV particle that does not comprise the modified capsid protein.

### Packaging Efficiency

According to some aspects, packaging efficiency of an AAV particle comprising a modified AAV capsid protein disclosed herein is altered relative to an AAV particle that does not comprise the modified capsid protein. Packaging efficiency of an AAV particle refers to the capability of a particular AAV capsid to encapsidate a particular viral genome. Packaging efficiency can be measured by one of ordinary skill in the art, such as by quantifying the ratio of capsids to viral genomes (see, e.g., Grimm, et al. Gene Ther. 6:1322-1330 (1999)).

In some embodiments, the packaging efficiency of an AAV particle comprising a modified AAV capsid protein as disclosed herein is higher than the packaging efficiency of an AAV particle that does not comprise the modified capsid protein. In some embodiments, the packaging efficiency of an AAV particle comprising a modified AAV capsid protein as disclosed herein is at least 5% higher (e.g., at least 10% higher, at least 15% higher, at least 20% higher, at least 25% higher, at least 30% higher, at least 35% higher, at least 40% higher, at least 50% higher, at least 60% higher, at least 70% higher, at least 80% higher, at least 90% higher, at least 100% higher, at least 150% higher, at least 200% higher, at least 250% higher, or more) than the packaging efficiency of an AAV particle that does not comprise the modified capsid protein. In some embodiments, the packaging efficiency of an AAV particle comprising a modified AAV capsid protein as disclosed herein is at least 1.5-fold higher (e.g., at least 2-fold higher, at least 2.5-fold higher, at least 3-fold higher, at least 3.5-fold higher, at least 4-fold higher, at least 4.5-fold higher, at least 5-fold higher, at least 5.5-fold higher, at least 6-fold higher, at least 6.5-fold higher, at least 7-fold higher, at least 7.5-fold higher, at least 8-fold higher, at least 8.5-fold higher, at least 9-fold higher, at least 9.5-fold higher, at least 10-fold higher, at least 10.5-fold higher, at least 11-fold higher, at least 11.5-fold higher, at least 12-fold higher, at least 12.5-fold higher, at least 13-fold higher, at least 13.5-fold higher, at least 14-fold higher, at least 14.5-fold higher, at least 15-fold higher, at least 15.5-fold higher, at least 16-fold higher, at least 16.5-fold higher, at least 17-fold higher, at least 17.5-fold higher, at least 18-fold higher, at least 18.5-fold higher, at least 19-fold higher, at least 19.5-fold higher, at least 20-fold higher, or more) than the packaging efficiency of an AAV particle that does not comprise the modified capsid protein.

In some embodiments, the packaging efficiency of an AAV particle comprising a modified AAV capsid protein as disclosed herein is lower than the packaging efficiency of an AAV particle that does not comprise the modified capsid protein. In some embodiments, the packaging efficiency of an AAV particle comprising a modified AAV capsid protein as disclosed herein is decreased by at least 5% (e.g., at least 10%, at least 15%, at least 20%, at least 25%, at least 30%, at least 35%, at least 40%, at least 50%, at least 60%, at least 70%, at least 75%, at least 80%, at least 85%, at least 90%, at least 91%, at least 92%, at least 93%, at least 94%, at least 95%, or more) relative to the packaging efficiency of an AAV particle that does not comprise the modified capsid protein.

In some embodiments, packaging efficiency of an AAV particle comprising a modified AAV capsid protein disclosed herein is not altered relative to an AAV particle that does not comprise the modified capsid protein.

In some embodiments, both the transduction efficiency and the packaging efficiency of an AAV particle comprising a modified AAV capsid protein as disclosed herein are modified (i.e., increased or decreased) relative to an AAV particle that does not comprise the modified capsid protein.

### Pharmaceutical Compositions

Any one of the AAV particles, capsid proteins, or nucleic acids disclosed herein may be comprised within a pharmaceutical composition comprising a pharmaceutically-acceptable carrier or may be comprised within a pharmaceutically-acceptable carrier. The term “carrier” refers to a diluent, adjuvant, excipient, or vehicle with which the AAV particle, capsid protein, or nucleic acid is comprised or administered to a subject. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases), and solutions or compositions thereof. Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of AAV particles to human subjects.

Typically, such compositions may contain at least about 0.1% of the therapeutic agent (e.g., AAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., AAV particle) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be designed.

### Methods of Contacting a Cell

According to some aspects, methods of contacting a cell with an AAV particle are provided herein. Methods of contacting a cell may comprise, for example, contacting a cell in a culture with a composition comprising an AAV particle. In some embodiments, contacting a cell comprises adding a composition comprising an AAV particle to the supernatant of a cell culture (e.g., a cell culture on a tissue culture plate or dish) or mixing a composition comprising an AAV particle with a cell culture (e.g., a suspension cell culture). In some embodiments, contacting a cell comprises mixing a composition comprising an AAV particle with another solution, such as a cell culture media, and incubating a cell with the mixture.

In some embodiments, contacting a cell with an AAV particle comprises administering a composition comprising an AAV particle to a subject or device in which the cell is located. In some embodiments, contacting a cell comprises injecting a composition comprising an AAV particle into a subject in which the cell is located. In some embodiments, contacting a cell comprises administering a composition comprising an AAV particle directly to a cell, or into or substantially adjacent to a tissue of a subject in which the cell is present.

In some embodiments, “administering” or “administration” means providing a material to a subject in a manner that is pharmacologically useful. In some embodiments, a rAAV particle is administered to a subject enterally. In some embodiments, an enteral administration of the rAAV particle is oral. In some embodiments, a rAAV particle is administered to the subject parenterally. In some embodiments, a rAAV particle is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a rAAV particle is administered to the subject by injection into the hepatic artery or portal vein.

In some embodiments, a composition of AAV particles is administered to a subject to treat a disease or condition. To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host organ, tissue, or cell. A therapeutically acceptable amount may be an amount that is capable of treating a disease, e.g., a neurological disease. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, a cell disclosed herein is a cell isolated or derived from a subject. In some embodiments, a cell is a mammalian cell (e.g., a cell isolated or derived from a mammal). In some embodiments, a cell is a human cell. In some embodiments, a cell is isolated or derived from a particular tissue of a subject, such as neurological tissue. In some embodiments, a cell is a central nervous system cell. In some embodiments, a cell is a neuron. In some embodiments, a cell is in vitro. In some embodiments, a cell is ex vivo. In some embodiments, a cell in in vivo. In some embodiments, a cell is within a subject (e.g., within a tissue or organ of a subject). In some embodiments, a cell is a primary cell. In some embodiments, a cell is from a cell line (e.g., an immortalized cell line). In some embodiments a cell is a cancer cell or an immortalized cell.

In some embodiments, “administering” or “administration” means providing a material to a subject in a manner that is pharmacologically useful.

In certain circumstances it will be desirable to deliver an AAV particle disclosed herein in a suitably formulated pharmaceutical composition disclosed herein either subcutaneously, intraocularly, intravitreally, subretinally, parenterally, intravenously (IV), intracerebro-ventricularly, intramuscularly, intrathecally (IT), intracisternally, orally, intraperitoneally, by oral or nasal inhalation, or by direct injection to one or more cells, tissues, or organs by direct injection. In some embodiments, the administration is a route suitable for systemic delivery, such as by intravenous injection. In some embodiments, the administration is a route suitable for local delivery, such as by intrathecal, intracerebro-ventricular, or intracisternal injection. In some embodiments, “administering” or “administration” means providing a material to a subject in a manner that is pharmacologically useful.

In some embodiments, the concentration of AAV particles administered to a subject may be on the order ranging from 10⁶ to 10¹⁴ particles/ml or 10³ to 10¹⁵ particles/ml, or any values therebetween for either range, such as for example, about 10⁶, 10⁷, 10⁸, 10⁹, 10¹⁰, 10¹¹, 10¹², 10¹³, or 10¹⁴ particles/ml. In some embodiments, AAV particles of a higher concentration than 10¹³ particles/ml are administered. In some embodiments, the concentration of AAV particles administered to a subject may be on the order ranging from 10⁶ to 10¹⁴ vector genomes (vgs)/ml or 10³ to 10¹⁵ vgs/ml, or any values therebetween for either range (e.g., 10⁶, 10⁷, 10⁸, 10⁹, 10¹⁰, 10¹¹, 10¹², 10¹³, or 10¹⁴ vgs/ml). In some embodiments, AAV particles of higher concentration than 10¹³ vgs/ml are administered. The AAV particles can be administered as a single dose, or divided into two or more administrations as may be required to achieve therapy of the particular disease or disorder being treated. In some embodiments, 0.0001 ml to 10 ml are delivered to a subject. In some embodiments, the number of AAV particles administered to a subject may be on the order ranging from 10⁶-10¹⁴ vgs/kg body mass of the subject, or any values therebetween (e.g., 10⁶, 10⁷, 10⁸, 10⁹, 10¹⁰, 10¹¹, 10¹², 10¹³, or 10¹⁴ vgs/kg). In some embodiments, the dose of AAV particles administered to a subject may be on the order ranging from 10¹²-10¹⁴ vgs/kg. In some embodiments, the volume of AAV composition delivered to a subject (e.g., via one or more routes of administration as described herein) is 0.0001 ml to 10 ml.

In some embodiments, a composition disclosed herein (e.g., comprising an AAV particle) is administered to a subject once. In some embodiments, the composition is administered to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a disease, disorder, or condition in the subject.

### Subjects

Aspects of the disclosure relate to methods for use with a subject, such as human or non-human primate subjects; with a host cell in situ in a subject; or with a host cell derived from a subject (e.g., ex vivo or in vitro). Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease or disorder that may be treated with gene therapy. In some embodiments, the subject has or is suspected of having a neurological disease or disorder. A neurological disease or disorder may be characterized by one or more mutation(s) in the genome that results in abnormal structure or function of one or more proteins associated with neurological development, health, maintenance and/or function. Exemplary neurological diseases and disorders include but are not limited to Alzheimer's Disease, amyotrophic lateral sclerosis, ataxia, Bell's palsy, brain tumors, cerebral aneurysm, epilepsy and seizure disorders, Guillain-Barre syndrome, headache, hydrocephalus, meningitis, multiple sclerosis, muscular dystrophy, Parkinson's disease, stroke, cluster headaches, migraine headaches, encephalitis, and myasthenia gravis. Neurological diseases and disorders can be characterized and identified, e.g., through laboratory tests and/or evaluation by a clinician. In some embodiments, the subject has or is suspected of having a disease involving cells of the central and/or peripheral nervous systems. In some embodiments, a nucleic acid isolated or derived from the subject (e.g., genomic DNA, mRNA, or cDNA from the subject) is identified via sequencing (e.g., Sanger or next-generation sequencing) to comprise a mutation (e.g., in a gene associated with neurological development, health, maintenance, or function).

### EXAMPLES

### Example 1. AAV Particles Comprising Modified Capsid Proteins with F535R Amino Acid Substitution

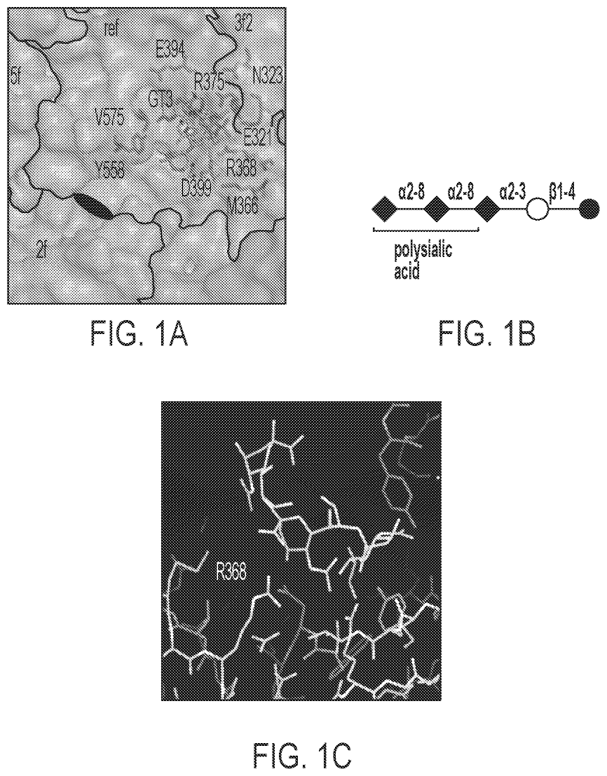

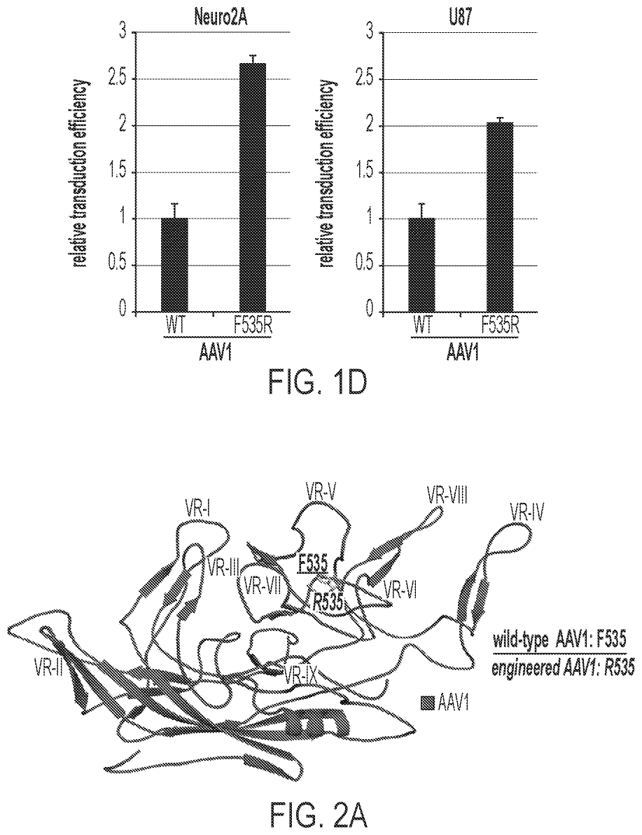

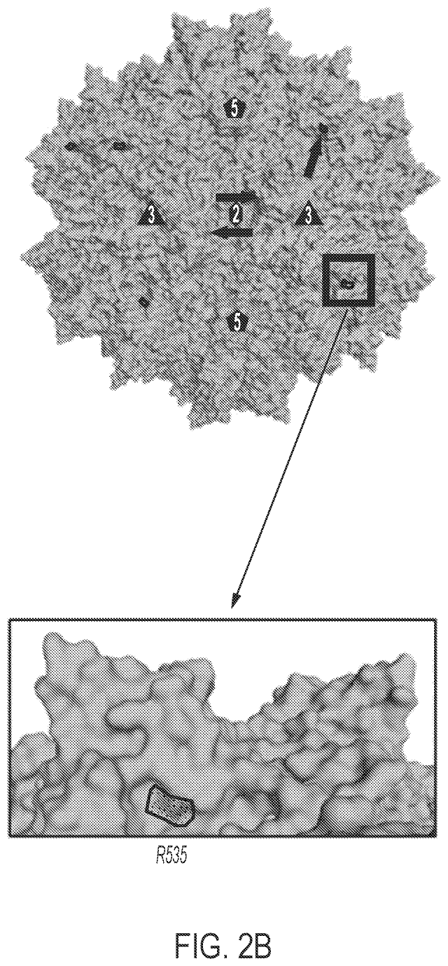

To generate AAV particles with enhanced transduction efficiency and delivery to cells of the central nervous system (CNS), wild-type AAV capsids were first compared with Minute virus of mice strain i (MVMi), which binds several glycans that are widespread in the brain. X-ray crystallography was used to determine the binding site (FIG. 1A) of GT3 glycan (FIG. 1B) to the capsid of MVMi. Residue R368 of MVMi (FIG. 1C) was identified to be important for the interaction between MVMi and GT3. This residue structurally aligns with residue F535 of AAV1 (FIG. 2A). F535 of wild-type AAV1 is surface-exposed (FIG. 2B), and therefore was identified as a candidate for modification to improve CNS targeting. To evaluate whether substitution of the phenylalanine at position 535 with an arginine residue (i.e., the amino acid at the corresponding position in the MVMi capsid) could affect interaction of AAV1 particles with neurons, AAV1 particles comprising F535R modified capsid proteins were produced, containing a vector genome encoding luciferase. Neuronal cells from the cell lines U87 and Neuro2a were incubated with wild-type AAV1 particles or AAV1-F535R particles, and transduction efficiency was measured based on luminescence of the cells following AAV incubation (FIG. 1D). The results demonstrate that the amino acid substitution F535R resulted in enhanced transduction efficiency of AAV1 particles (˜2-3-fold increase) relative to wild-type.

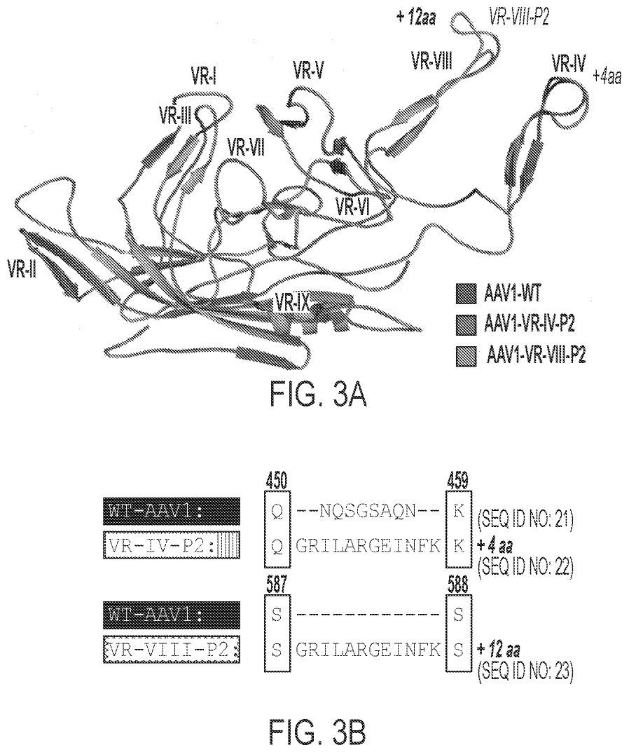

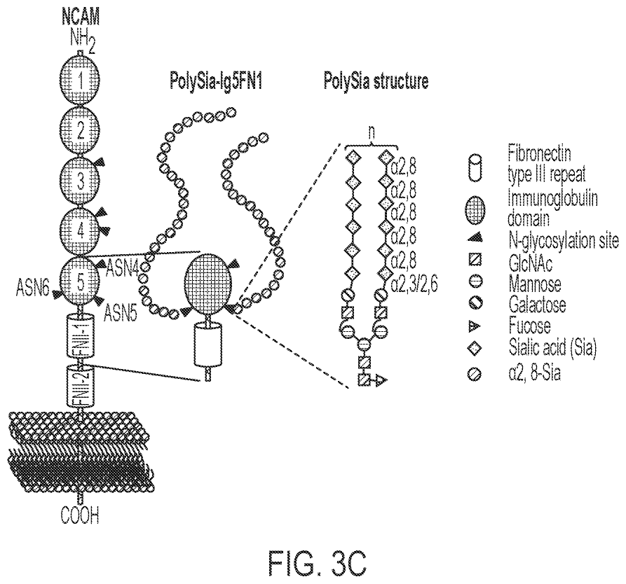

As an alternative approach to achieve improved neuronal gene transfer, neural cell adhesion molecule (NCAM) was identified as a candidate target to which AAV particles might be directed. Non-native peptides were inserted into surface variable regions (VRs) of AAV1 capsid proteins and tested for changes in transduction efficiency of AAV particles comprising the modified capsid proteins. P2 peptide having the amino acid sequence GRILARGEINFK (SEQ ID NO: 15) was identified as a lead candidate and was further tested substituted into each of the VRs of AAV1. VR-IV and VR-VIII were identified as good locations for P2 insertion, so modified capsid proteins and AAV particles comprising them were produced, termed “VR-IV-P2” and “VR-VIII-P2” for P2 inserted in the VR-IV loop and P2 inserted in the VR-VIII loop, respectively. Ribbon diagrams of these two modified capsid proteins are shown in FIG. 3A, and the sequences at the VR-IV and VR-VIII loci are shown in FIG. 3B. Modified AAV particles comprising these modified capsid proteins were generated to target NCAM as expressed in the brain (FIG. 3C).

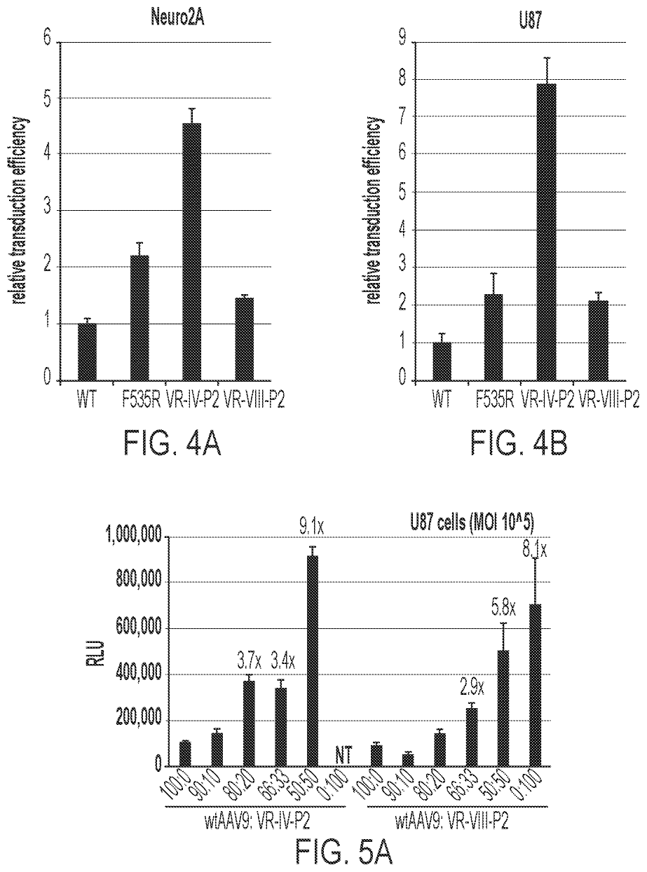

Neuro2a and U87 cells were incubated with wild-type, F535R, VR-IV-P2, and VR-VIII-P2 AAV1 particles, and transduction efficiency was measured (FIGS. 4A and 4B). The results demonstrate that insertion of the P2 peptide within capsid proteins can increase transduction efficiency of AAV1 particles in neuronal cells (˜4-8-fold for VR-IV-P2 modified AAV particles and ˜1.5-2-fold for VR-VIII-P2 modified AAV particles).

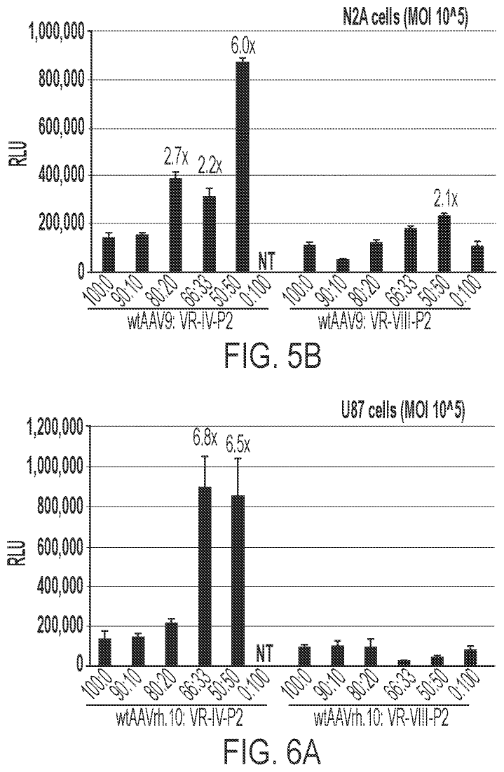

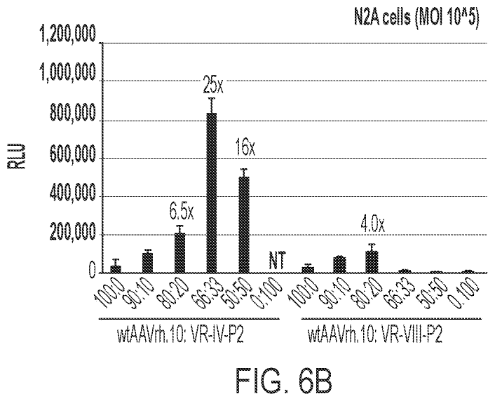

AAV9 and AAVrh10 particles comprising P2 insertions at VR-IV and VR-VIII were also produced. It was observed that AAV9 particles comprising P2 insertions within their capsid proteins demonstrated lower production efficiency, so chimeric AAV9 and AAVrh10 particles comprising both wild-type and P2-inserted capsid proteins at various ratios were produced and tested for their transduction efficiency in U87 and Neuro2a (“N2A”) cells (FIGS. 5A, 5B, 6A, and 6B). The results demonstrate that AAV9 and AAVrh10 particles comprising capsid proteins with P2 peptide insertions have varying transduction efficiency in neuronal cells depending on the ratio of wild-type to P2-modified capsid proteins within the AAV particle. The effect was also dependent upon the site of insertion of the P2 peptide (VR-IV versus VR-VIII).

### Example 2. Immunogenicity of Modified AAV Particles

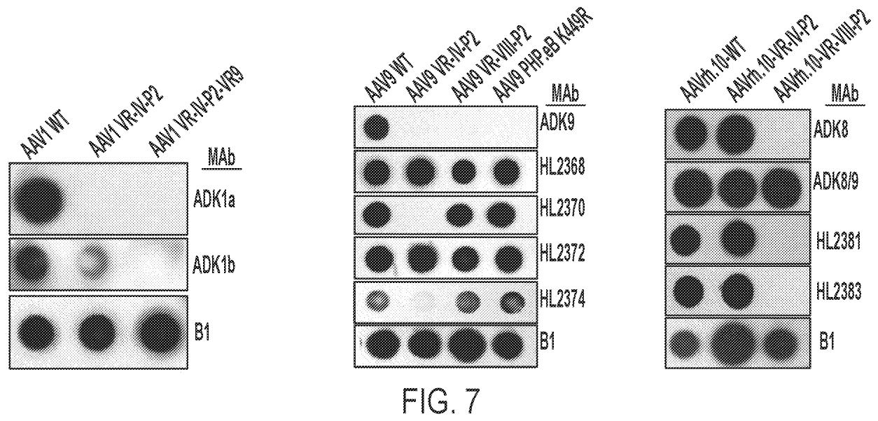

The immunogenicity of AAV particles comprising modified capsid proteins was tested. AAV1, AAV9, and AAVrh10 particles were produced comprising P2 peptide inserted in VR-IV or VR-VIII. 10¹⁰ capsids of each variant were loaded onto a nitrocellulose membrane and tested against monoclonal antibodies (MAbs) with known binding to AAV capsids. The results demonstrate that the changes introduced into the AAV capsids modified the antigenicity by eliminating an epitope bound by neutralizing antibodies (FIG. 7). This facilitates the utilization of these modified AAV particles in the presence of neutralizing antibodies, such as in vivo in a subject with anti-AAV neutralizing antibodies.

### Example 3. Cryo-Electron Microscopy of AAV Particles

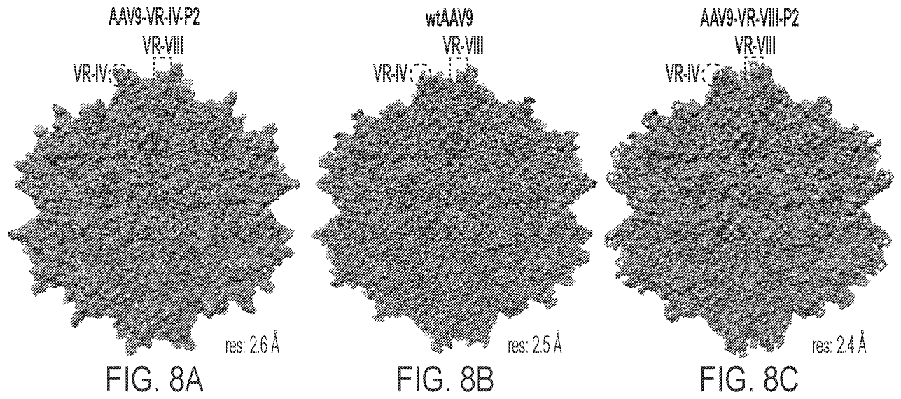

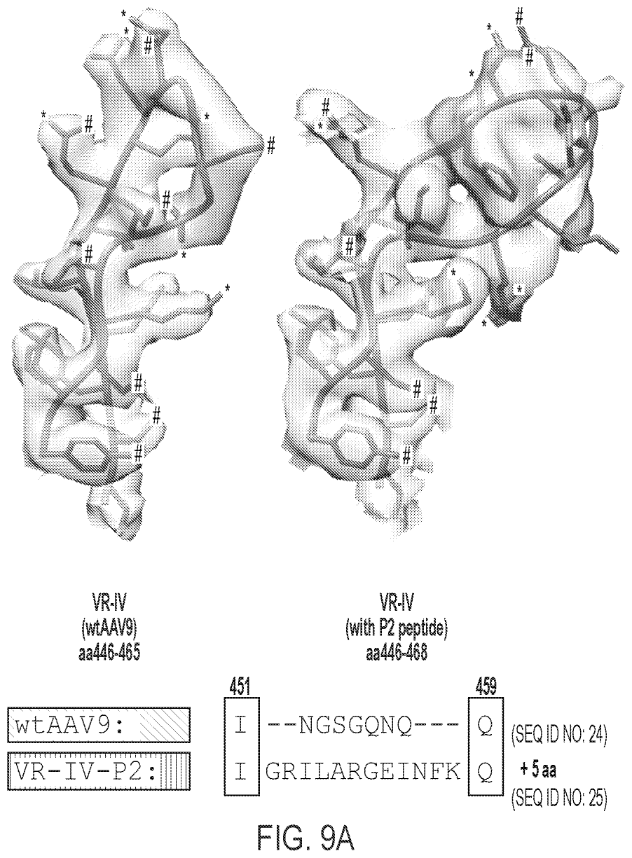

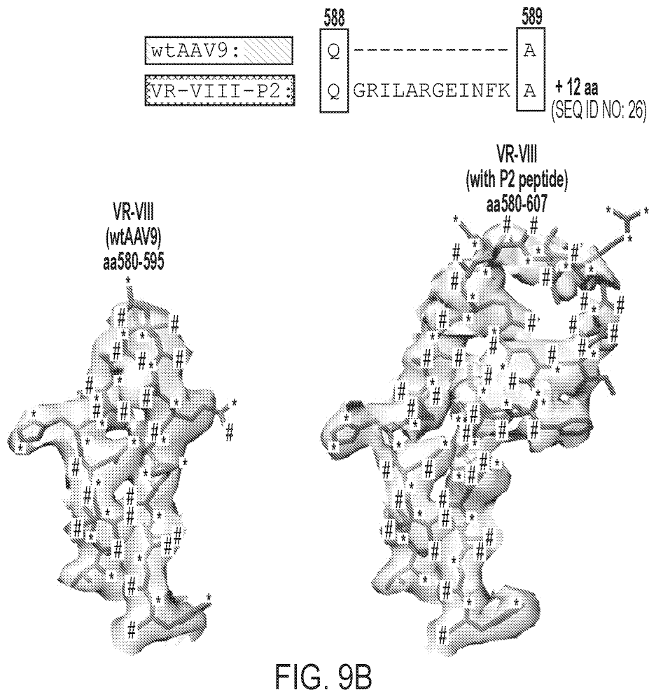

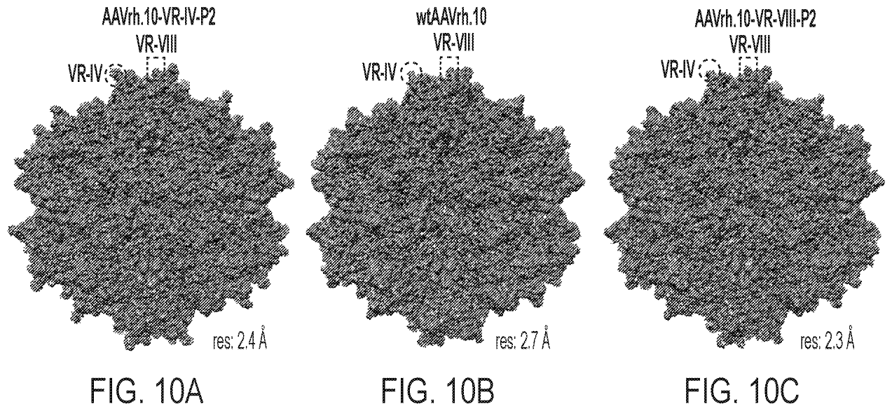

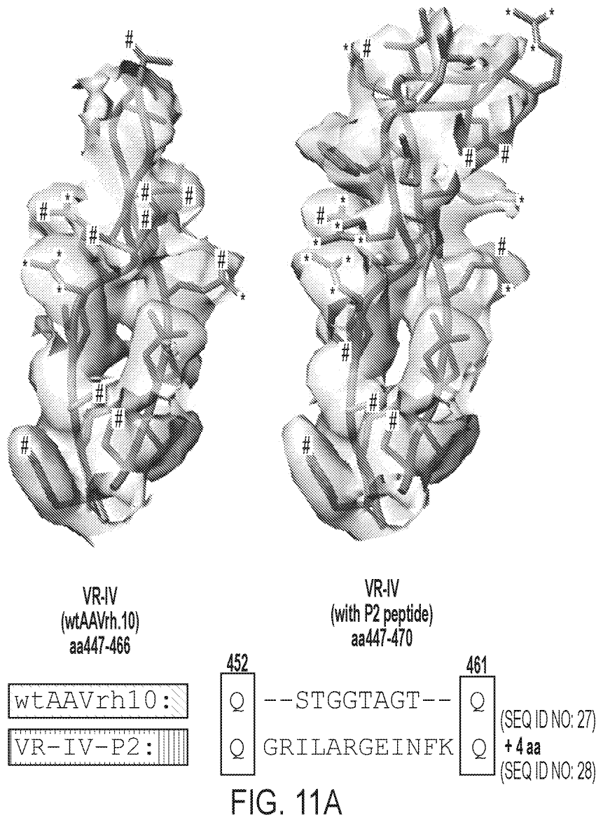

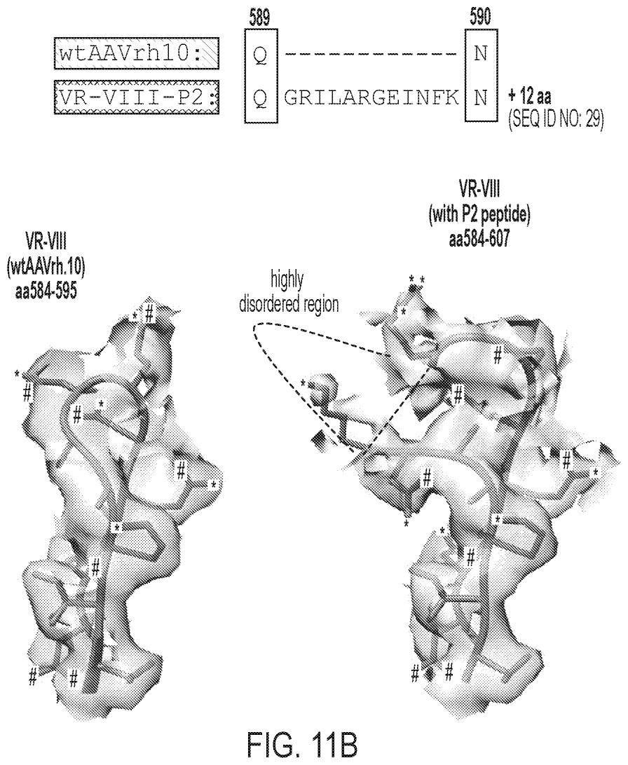

The structure of AAV particles comprising modified capsid proteins was elucidated using cryo-electron microscopy (cryo-EM). AAV9 and AAVrh10 capsid proteins and particles were produced comprising P2 peptide inserted in VR-IV or VR-VIII. Cryo-EM was conducted on wild-type and P2 variants, and density maps of the capsids (FIGS. 8A-8C and 10A-10C) and atomic structures of VR-IV and VR-VIII (FIGS. 9A-9B and 11A-11B) were constructed from the cryo-EM data.

### Additional Embodiments

The following embodiments are within the scope of the present disclosure. Furthermore, the disclosure encompasses all variations, combinations, and permutations of these embodiments in which one or more limitations, elements, clauses, and descriptive terms from one or more of the listed embodiments is introduced into another listed embodiment in this section. For example, any listed embodiment that is dependent on another embodiment can be modified to include one or more limitations found in any other listed embodiment in this section that is dependent on the same base embodiment. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the invention or aspects of the invention consist, or consist essentially of, such elements and/or features. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A modified adeno-associated virus (AAV) capsid protein comprising a non-native peptide within loop IV or loop VIII of the capsid protein, wherein the non-native peptide comprises an amino acid sequence selected from GRILARGEINFK (SEQ ID NO: 15), ASKKPKRNIKA (SEQ ID NO: 16), AKKMWKKTW (SEQ ID NO: 17), GEISVGESKFFL (SEQ ID NO: 18), KHIFSDDSSELTIRNVDKNDE (SEQ ID NO: 19), and SIHLKVFAK (SEQ ID NO: 20).

2. The modified AAV capsid protein of claim 1, wherein the non-native peptide is positioned between amino acids corresponding to Q450 and K459 of the wild-type AAV1 capsid protein as set forth in SEQ ID NO: 1.

3. The modified AAV capsid protein of claim 1 or 2, wherein the non-native peptide replaces the amino acids corresponding to positions 451 to 458 of the wild-type AAV1 capsid protein as set forth in SEQ ID NO: 1.

4. The modified AAV capsid protein of claim 1, wherein the non-native peptide is positioned between amino acids corresponding to S587 and S588 of the wild-type AAV1 capsid protein as set forth in SEQ ID NO: 1.

5. A modified AAV capsid protein comprising an arginine amino acid at a position corresponding to F535 of the wild-type AAV1 capsid protein as set forth in SEQ ID NO: 1.

6. The modified AAV capsid protein of any one of claims 1 to 5, wherein the modification results in increased binding of the AAV capsid protein to glycans relative to a corresponding unmodified AAV capsid protein.

7. The modified AAV capsid protein of any one of claims 1 to 6, wherein the modification results in increased binding of the AAV capsid protein to polysialic acid relative to a corresponding unmodified AAV capsid protein.

8. The modified AAV capsid protein of claim 7, wherein the polysialic acid is attached to a neuronal cell adhesion molecule.

9. The modified AAV capsid protein of any one of claims 1-8, wherein the modification results in increased binding of the AAV capsid protein to a cell of the central nervous system relative to a corresponding unmodified AAV capsid protein.

10. The modified AAV capsid protein of claim 8, wherein the cell of the central nervous system is a neuronal cell.

11. The modified AAV capsid protein of claim 10, wherein the neuronal cell is a brain neuronal cell.

12. The modified AAV capsid protein of any one of claims 1 to 11, wherein the AAV capsid protein is an AAV1, AAV9, AAVrh10 serotype capsid protein, or a combination thereof.

13. An AAV particle comprising the modified AAV capsid protein of any one of claims 1 to 12.

14. The AAV particle of claim 13, wherein the AAV particle is less immunogenic relative to a corresponding AAV particle not comprising the modified capsid protein.

15. The AAV particle of claim 13 or 14, wherein the AAV particle is less susceptible to binding by a neutralizing antibody than a corresponding wild-type AAV particle not comprising the modified capsid protein.

16. The AAV particle of claim 15, wherein the neutralizing antibody is an ADKla, ADK1b, ADK9, HL2370, HL2374, ADK8, HL2381, or HL2383 antibody.

17. The AAV particle of any one of claims 13 to 16, wherein the transduction efficiency of the AAV particle in a cell of the central nervous system is increased relative to a corresponding AAV particle not comprising the modified capsid protein.

18. The AAV particle of any one of claims 13 to 17, further comprising a nucleic acid segment encoding a therapeutic or diagnostic agent operably linked to a promoter.

19. The AAV particle of claim 18, wherein the promoter is a GAD65 promoter, a SYN promoter, a GFAP promoter, an INA promoter, an NES promoter, an MOBP promoter, an MBP promoter, a TH promoter, or a FOXA2/HNF30 promoter.

20. The AAV particle of claim 18 or claim 19, wherein the therapeutic or diagnostic agent is IDUA, IDS, SGSH, NAGLU, TPP1/CLN2, CLN3, CLN6, ARSA, ASPA, AADC, NTN, GDNF, NGF, APOE2, or SMN.

21. The AAV particle of any one of claims 13 to 20, wherein the AAV particle comprises the modified AAV capsid protein and an unmodified wild-type capsid protein.

22. The AAV particle of claim 21, wherein the ratio of unmodified wild-type capsid proteins to modified capsid proteins is about 95:5, 90:10, 85:15, 80:20, 75:25, 70:30, 66:33, 50:50; 33:66, 25:75, 20:80, 15:85, 10:90, or 5:95.

23. The AAV particle of any one of claims 13 to 22, wherein the particle is an AAV1, AAV9, or AAVrh10 serotype particle, or a combination thereof.

24. A method of delivering a therapeutic or diagnostic agent to a cell, the method comprising contacting a cell with the AAV particle of any one of claims 18 to 23 in an amount sufficient to promote internalization of the AAV particle in the cell, optionally wherein the cell is a central nervous system cell.

25. A method of treating a subject, the method comprising administering the AAV particle of any one of claims 13 to 23 to a subject in need thereof in an amount sufficient to alleviate one or more symptoms in the subject.

26. The method of claim 25, wherein the subject is suspected of having or has been diagnosed as having a neurological disease, disorder, or condition.

27. The method of claim 25 or 26, wherein the subject is a mammal.

28. The method of any one of claims 25 to 27, wherein the subject is a human.


SEQUENCE LISTING (29 sequences)

SEQ ID NO: 1 - protein, 736 aa, adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, adeno-associated virus 2 [sequence omitted]

SEQ ID NO: 3 - protein, 736 aa, adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 734 aa, adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 5 - protein, 724 aa, adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 736 aa, adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 737 aa, adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 8 - protein, 738 aa, adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 738 aa, adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 11 - protein, 733 aa, adeno-associated virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 12 - protein, 742 aa, adeno-associated virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 13 - protein, 733 aa, adeno-associated virus 13
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 14 - protein, 738 aa, adeno-associated virus 14; identical to SEQ ID NO: 10

SEQ ID NO: 15 - protein, 12 aa, synthetic construct
GRILARGEINFK

SEQ ID NO: 16 - protein, 11 aa, synthetic construct
ASKKPKRNIKA

SEQ ID NO: 17 - protein, 9 aa, synthetic construct
AKKMWKKTW

SEQ ID NO: 18 - protein, 12 aa, synthetic construct
GEISVGESKFFL

SEQ ID NO: 19 - protein, 21 aa, synthetic construct
KHIFSDDSSELTIRNVDKNDE

SEQ ID NO: 20 - protein, 9 aa, synthetic construct
SIHLKVFAK

SEQ ID NO: 21 - protein, 10 aa, adeno-associated virus 1
QNQSGSAQNK

SEQ ID NO: 22 - protein, 14 aa, synthetic construct
QGRILARGEINFKK

SEQ ID NO: 23 - protein, 14 aa, synthetic construct
SGRILARGEINFKS

SEQ ID NO: 24 - protein, 9 aa, adeno-associated virus 9
INGSGQNQQ

SEQ ID NO: 25 - protein, 14 aa, synthetic construct
IGRILARGEINFKQ

SEQ ID NO: 26 - protein, 14 aa, synthetic construct
QGRILARGEINFKA

SEQ ID NO: 27 - protein, 10 aa, synthetic construct
QSTGGTAGTQ

SEQ ID NO: 28 - protein, 14 aa, synthetic construct
QGRILARGEINFKQ

SEQ ID NO: 29 - protein, 14 aa, synthetic construct
QGRILARGEINFKN